(19) United States
(12) Patent Application Publication
(10) Pub. No.: US 2025/0009755 A1
(43) Pub. Date: Jan. 9, 2025

(54) COMPOSITIONS AND METHODS FOR THE TREATMENT OF OCULAR DISEASES AND INJURIES

(71) Applicant: Ohio State Innovation Foundation, Columbus, OH (US)

(72) Inventors: Andre PALMER, Columbus, OH (US); Katelyn Elizabeth REILLY, Columbus, OH (US); Megan ALLYN, Columbus, OH (US)

(21) Appl. No.: 18/710,916

(22) PCT Filed: Nov. 16, 2022
(86) PCT No.: PCT/US22/79971

§ 371 Date: May 16, 2024

Related U.S. Application Data
(60) Provisional application No. 63/413,438, filed on Oct. 5, 2022.
(60) Provisional application No. 63/280,095, filed on Nov. 16, 2021.

(51) Int. Cl. A61K 31/555; A61K 9/00; A61K 45/06; A61K 47/64; A61P 27/02

(52) U.S. Cl. CPC A61K 31/555; A61K 9/0048; A61K 45/06; A61K 47/64; A61K 47/643; A61P 27/02

(57) ABSTRACT

Disclosed are ocular therapeutic compositions as well as methods for treating ocular diseases and injuries using these compositions.

### CROSS REFERENCE TO RELATED APPLICATIONS

This application claims benefit of U.S. Provisional Application No. 63/280,095, filed Nov. 16, 2021, and U.S. Provisional Application No. 63/413,438, filed Oct. 5, 2022, each of which is hereby incorporated by reference in its entirety.

### STATEMENT REGARDING FEDERALLY SPONSORED RESEARCH OR DEVELOPMENT

This invention was made with government support under grant/contract numbers R01 HL 138116, R01 HL 126945, and R01 EB021926 awarded by National Institutes of Health. The government has certain rights in the invention.

### INCORPORATION BY REFERENCE OF SEQUENCE LISTING

The content of the XML file named “103361-169WO1.xml,” which was created on Nov. 16, 2022, and is 8,012 bytes in size, is hereby incorporated by reference in its entirety.

### BACKGROUND

Age related macular degeneration (AMD) is the leading cause of irreversible vision loss in the United States and many other industrialized countries. “Dry” AMD is the most common type of macular degeneration and affects 90% of the people who have the condition. The dry form is characterized by the formation of drusen within the macula, a specialized structural region of the retina which capture the light that enters the eye. Typically, drusen are formed under the retinal pigment epithelial (RPE) cells and its presence is thought to lead to atrophy of photoreceptors due to a breakdown or thinning of the RPE layer of that supports the photoreceptor cells. Ultimately, dry AMD can progress to geographic atrophy (GA), which is characterized by localized sharply demarcated atrophy of outer retinal tissue, retinal pigment epithelium and choriocapillaris. This tissue damage can result in blind spots in the patient's central vision. The persistence of drusen within the retina can also lead to a persistent inflammatory reaction and results in a cascade of secondary responses that eventually can lead to wet AMD.

The “wet” form of AMD is characterized by an abnormal outgrowth of blood vessels from the vasculature situated behind the retina in a process that is often referred to as choroidal neovascularization (CNV). While not as prevalent as the dry form, it has a more rapid onset and is more severe phenotype, often leading to reduction of a substantial portion of the visual field.

The current standard of care for wet AMD is Ranibizumab (RAN), a monoclonal antibody fragment with strong affinity to the vascular endothelial growth factor-A (VEGF-A), a molecular moiety secreted from cells and known to cause the formation or growth of nascent blood vessels. RAN binds to and inhibits the biologic activity of VEGF-A, thereby preventing the interaction of VEGF-A with its receptors (VEGFR1 and VEGFR2) on the surface of endothelial cells. This results in a reduction in endothelial cell proliferation, less vascular leakage, and a reduction in new blood vessel formation characteristic of CNV.

The ocular half-life of RAN, however, is only nine days following intravitreal injection, thus therapeutic doses must be administered monthly to patients to remain effective at suppressing vascular proliferation. To address this shortcoming, Genentech has developed SUSVIMO, an intraocular implant that delivers a therapeutically effective dose of RAN over a period of 24 weeks.

Although RAN is effective to stabilize visual acuity in nearly 95% of patients, improved vision was noted in only 29%-40% of patients. RAN acts as a molecular sponge to mop-up secreted VEGF-A. Inefficiencies in this process may be one reason why vision is only stabilized, not improved in most patients. In other words, it treats the symptoms but not the cause.

Other therapies for AMD have been developed targeting VEGF, including bevacizumab (a monoclonal antibody that inhibits VEGF-A), aflibercept (a recombinant fusion protein including VEGF-binding portions from the extracellular domains of human VEGF receptors 1 and 2), faricimab (a bispecific monoclonal antibody targeting both VEGF and angiopoietin 2), and brolucizumab (a monoclonal antibody that inhibits VEGF-A).

The principal drawback with existing monoclonal antibody wet AMD therapies is the requirement for frequent, continuous treatment, typically involving monthly injections into the eye. Combined with a rapidly aging population and correspondingly low numbers of clinicians who are qualified to administer intravitreal injections, application of this therapy This has placed enormous strain on healthcare systems. Thus there is clearly a need for longer lasting treatments and/or treatments that can reverse the symptoms. Alternative treatments for wet AMD have been similarly unsatisfactory, also as a result of their frequency of administration, but as well as their side effects or poor efficacy

One of the newer drugs to commence clinical trials is that of the VEGF Trap Eye (VTE) which incorporates the second binding domain of the VEGFR-1 receptor and the third domain of the VEGFR2 receptor 1. By fusing these extracellular protein sequences to the Fc segment of a human IgG backbone, developers have created a chimeric protein with a very high VEGF binding affinity. As well as binding all isomers of the VEGF-A family, it also binds VEGF-B and placental growth factor.

Given the fact that the chimera protein still has a relatively short half-life, VTE however must still be regularly administered—every 2 months.

AAV2-sFLT01 is a gene therapy vector that expresses a modified soluble Flt1 receptor coupled to a human IgG1 Fc. As a high affinity VEGF binding protein, AAV2-sFLT01 functions to neutralize the pro-angiogenic activities of VEGF for treatment of wet AMD via an intravitreal injection. (Wasworth et al. Molecular Therapy vol. 19 no 2 Feb. 2011; 326-334) The use of an AAV vector is expected to ensure long-term expression, lasting for many months or even years, from a single injection. However, in order to accommodate the sFLT01 and IgG1 Heavy Chain Fc fusion protein, single stranded AAV must be used, which in turn requires high quantities of vector for efficient transduction and thus increases the risk of an immune response to the viral capsid proteins. Moreover, a high prevalence of the normal adult population has been exposed to serotype 2 variant of AAV, and may have pre-existing immunity against it.

The molecule PF-04523655 is a 19 nucleotide siRNA that inhibits the expression of the hypoxia-inducible gene RTP801 (Nguyen et al. Ophthalmology. 2012 September; 119(9):1867-73). In clinical studies conducted to date, it has been found to prevent neovascularization and vessel leakage, although does so via a different pathway than VEGF It has been demonstrated that the siRNA only persists in the eye for several weeks, meaning that like so many of the other existing and developing therapies, patients will require regular intravitreal injections for treatment. A failure to do so with many treatments has seen a continued loss of visual acuity, and a progression of degeneration.

More generally, siRNA-based approaches for treating and managing AMD have failed. Although initial pre-clinical experimental results were encouraging, it was subsequently demonstrated that mode of action of these molecules was not through a sequence specific RNAi-based mechanism, but rather through induction of a non-specific interferon response mediated by the interaction of siRNAs with Toll-like receptor TLR3 (Kleinmann et al 2008) Toll-like receptors are transmembrane proteins that play a key role in the innate immune system. Often positioned on either the cell surface or on intracellular vesicles such as the endosome, some family members of this family recognize double stranded RNA, not normally present in the endogenous cell, as foreign substance and triggers a cascade of molecule responses. This leads to interferon activation, which has a transitory therapeutic effect in mouse models. However interferon has a much lower efficacy in humans which explains the poor efficacy of this treatment in human clinical testing.

Retinostat is an equine infectious anemia virus (EIAV) based lentivirus vector expressing angiostatin and endostatin, both of which are naturally occurring angiogenesis inhibitors in the ocular compartment. Endostatin blocks VEGF signaling, reduces vascular permeability, decreases cell matrix adhesion and promotes endothelial cell apoptosis. Angiostatin prevents endothelial cell proliferation and migration. The genes are delivered via a subretinal injection and inhibit the formation of new blood vessels Sub-retinal delivery however requires an intensive surgical procedure, which, unlike intravitreal delivery, does not lend itself to outpatient treatments or treatment at a local doctor.

Complement inhibition is also being explored as a strategy for treating geographic atrophy. In the Phase 2 FILLY study, intravitreal injection of the C3 inhibitor pegcetacoplan (APL-2; Apellis Pharmaceuticals; Crestwood, KY, USA) significantly reduced GA growth rate. In the Phase 2/3 GATHER1 study, intravitreal injections of the anti-C5 aptamer avacincaptad pegol/ARC1905 (Zimura®; IVERIC Bio [formerly, Ophthotech], Cranbury, NJ, USA) significantly reduced GA growth rate.

Despite the large amount of development activity in the field of AMD therapeutics, there remains a need to create more effective therapies that are also patient friendly with respect to side effects, the mode of treatment and the frequency thereof.

### SUMMARY

Age-related macular degeneration (AMD) is a retinal neurodegeneration disorder affecting central vision and can lead to permanent blindness if left untreated. The local para-inflammatory response induced by reactive oxygen species (ROS) that further contributes to the excess expression of vascular endothelial growth factor (VEGF) is considered one of the main causes of AMD. Current treatment relies on using anti-VEGF to inhibit choroidal neovascularization (CNV) in the retina and maintain the active function of photoreceptors and retinal pigment epithelial (RPE) cells. However, medications used for AMD, such as bevacizumab, aflibercept, and ranibizumab, cannot prevent or cure the disease. Instead, these therapies target angiogenesis without targeting the upstream inflammatory factors, significantly limiting their therapeutic effect. Moreover, these anti-angiogenetic factors have a relatively short half-life and poor sustained release in the eye, requiring monthly injections that increase cost and burden on the healthcare system, lowers patient compliance, and potentially leads to unsatisfactory clinical outcomes.

To address these current limitations, described herein is an antioxidant nanoparticle (NP)-based approach to improve the treatment of AMD by 1) effectively scavenging disease-inducing ROS, 2) releasing anti-angiogenesis (e.g., anti-VEGF) or anti-inflammatory (e.g., heme-albumin) therapeutics controllably to treat the disease, and 3) extending therapeutic life-span and drug release to reduce injection frequency. Without wishing to be bound by theory, it is believed that human serum albumin (HSA) bound to heme (heme-albumin) will induce retinal cells to overexpress heme-oxygenase-1 (HO-1), reducing inflammatory markers.

Accordingly, provided herein are ocular therapeutic compositions that comprise a therapeutically effective amount of heme to treat or prevent an ophthalmological disorder in a subject in need thereof.

In some embodiments, the heme can comprise free heme. In other embodiments, the heme can comprise a heme conjugate (e.g., heme covalently or non-covalently associated with a protein or peptide). In some embodiments, the heme conjugate can comprise beme-human serum albumin (heme-HSA). In some embodiments, the heme conjugate can comprise methemoglobin. In some cases, the methemoglobin can be conjugated to HSA. In some embodiments, the heme conjugate can comprise polymerized methemoglobin. In some embodiments, the beme conjugate can comprise a methemoglobin-haptoglobin conjugate.

In some embodiments, the ocular therapeutic composition can further comprise a delivery vehicle for delivery of the heme to the eye. The drug delivery vehicle can comprise, for example, gels (e.g., hydrogels such as an alginate hydrogel or a hyaluronic acid hydrogel), capsules, particles, or other vehicles that modulate solubility, delivery, stability, and or release of the heme within the eye. In some embodiments, the delivery vehicle can comprise a population of particles formed from a biocompatible polymer, and wherein the heme is associated with the particles.

In some embodiments, the beme can be encapsulated within the particles. In some embodiments, the heme can be non-covalently associated with the particles, such as adsorbed to the surface of the particles (or within pores on the surface of the particles).

In some embodiments, the particles can comprise stimuli-responsive particles (e.g., particles which undergo a change in response to a stimulus, such as a change in pH, a change in temperature, or the presence of other species, triggering release of their therapeutic cargo). In certain embodiments, the heme can be released upon exposure of the particles to reactive oxygen species (e.g., the particles are responsive to the presence of ROS).

The biocompatible polymer can comprise any suitable biocompatible polymer. In some embodiments, the biocompatible polymer can comprise a biodegradable polymer. In certain embodiments, the biocompatible polymer can comprise polydopamine

In some embodiments, the particles can comprise nanoparticles. In some embodiments, the population of particles can have an average particle size of from about 10 nm to about 1000 nm, such as from about 100 nm to about 400 nm, from about 100 nm to about 200 nm, from about 120 nm to about 270 nm, or from about 120 nm to about 170 nm. In certain embodiments, the population of particles can have an average particle size of about 150 nm, about 175 nm, or about 200 nm.

In certain embodiments, the particles can be further coated with a coating polymer, such as alginate, a polyalkylene oxide (e.g., PEG), a polyester (e.g., polylactic acid, polyglycolic acid, PLGA, polycaprolactone), polyvinyl alcohol, copolymers thereof, and combinations thereof.

In some embodiments, the composition can further comprise an additional active agent (in addition to the heme). When a delivery vehicle is present in the composition, the delivery vehicle can further provide for delivery of the additional active agent to the eye. For example, in the case of compositions comprising a delivery vehicle comprising a population of particles formed from a biocompatible polymer, the additional active agent can also be incorporated in the particles. In some embodiments, the additional active agent can be encapsulated within the particles. In some embodiments, the additional active agent can be non-covalently associated with the particles, such as adsorbed to the surface of the particles (or within pores on the surface of the particles). The additional active agent can comprise an ophthalmic drug, such as an anti-glaucoma agent, an anti-angiogenesis agent, an anti-vascular endothelial growth factor (VEGF) agent, an anti-infective agent, an anti-inflammatory agent, a growth factor, an immunosuppressant agent, an anti-allergic agent, complement inhibitor, or any combinations thereof.

Also provided are methods of treating an ophthalmological disorder in a subject in need thereof comprising contacting the eye of the subject with a therapeutically effective amount of an ocular therapeutic composition described herein.

In some embodiments, contacting the eye of the subject can comprise topical application of the composition to the eye of the subject (e.g., in the form of an eye drop). In some embodiments, contacting the eye of the subject can comprise injecting the composition into the eye of the subject. Injecting into the eye of the subject can comprise injecting into the vitreous chamber of the eye. In some examples, injecting into the eye of the subject can comprise an intravitreal injection, a subconjunctival injection, a subtenon injection, a retrobulbar injection, or a suprachoroidal injection. The composition can also be delivered via other suitable means, such as via microneedle injection, or by passage through the cornea.

The ophthalmological disorder can comprise, for example, acute macular neuroretinopathy; Behcet's disease; neovascularization, including choroidal neovascularization; diabetic uveitis, histoplasmosis; infections, such as fungal or viral-caused infections; macular degeneration, such as acute macular degeneration (AMD), including wet AMD, non-exudative AMD and exudative AMD; a retinal degenerative disease, such as geographic atrophy; edema, such as macular edema, cystoid macular edema and diabetic macular edema; multifocal choroiditis, ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic ophthalmia, Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, nonretinopathy diabetic retinal dysfunction, retinitis pigmentosa, a cancer, and glaucoma. In certain embodiments, the ophthalmological disorder is AMD, such as dry AMD.

### DESCRIPTION OF DRAWINGS

FIG. 1 schematically illustrates how PDA nanoparticles can be loaded with different therapeutics, including large antibodies and proteins. In this example, heme-albumin serves as the cargo. PDA has the ability to scavenge ROS, which also triggers release of the heme-albumin cargo, treating two mechanisms of retinal degenerative diseases while sustaining release.

FIG. 2A shows microscopy images of PDA nanoparticles and therapeutic loaded nanoparticles.

FIG. 2B shows that ROS levels of ARPE-19 cells were reduced by PDA nanoparticles in the presence of hydrogen peroxide oxidative stressor. Intracellular stress was indicated by dichlorofluorescein with green fluorescence.

FIG. 2C shows the degradation of nanoparticles stimulated by ROS.

FIG. 2D shows the ROS responsive release of labeled protein therapeutic FITC-BSA (bovine serum albumin) from PDA nanoparticles under various concentrations of hydrogen peroxide.

FIG. 3A is a plot showing the incorporation of heme into HSA. Size exclusion HPLC of HSA and heme-albumin. Fluorescence emission was monitored at 330 nm (excitation at 285 nm), and showed quenching of the fluorescence emission by heme in heme-albumin, whereas HSA has a strong emission at 330 nm. Inlay-X-ray crystal structure of heme-HSA (PDB: 1N5U).

FIG. 3B is a plot showing the ARPE-19 cell viability after 24 hours of incubation with 2:1 molar ratio heme-albumin loaded nanoparticles.

FIGS. 4A-4D illustrate AMD and the therapeutic strategies described herein. FIG. 4A shows drusen on the retina in dry AMD from oxidative damage. FIG. 4B illustrates excess VEGF expression causes choroidal neovascularization (CNV) and vision loss in wet AMD. FIG. 4C shows how anti-VEGF is used clinically to slow AMD progression. FIG. 4D illustrates proposed stimuli-responsive NPs to sustain release of heme-albumin and anti-VEGF to target ROS and CNV.

FIG. 5 illustrates that monthly bolus IVT injections use excess therapeutic and have concentration spikes due to short half-life. Controlled release can maintain vitreous levels >10× IC50.

FIG. 6A shows microscopy images of PDA NPs and anti-VEGF loaded NPs.

FIG. 6B shows that NP degradation is accelerated by ROS.

FIG. 6C shows NPs (green) enter cells within 24 hours of incubation.

FIG. 7 shows the effect of ROS on FITC-BSA and anti-VEGF release from PDA NPs. A higher amount of H₂O₂ led to faster release of therapeutics. NPs were degraded by 2-3 months in the presence of higher levels of H₂O₂. Data are average±standard deviation (n=3).

FIG. 8 illustrates ARPE-19 cell viability after treatment with PDA NPs as assessed by MTS assay. No significant cell death was observed at NP concentrations between 0 to 50 μg/mL (*p≥0.05).

FIG. 9 illustrates that PDA NPs exhibit the ability to scavenge and reduce intracellular ROS induced by H₂O₂, as shown by green dichlorofluorescein (DCFH-DA) probe (top). H₂O₂ was used to induce angiogenesis in HUVECs (bottom). A significant decrease in fluorescence (Calcein AM) and tubule length was seen in groups tested with PDA NPs and bevacizumab control (n=3, p<0.05). Scale bar=300 μm.

FIGS. 10A-10D show the incorporation of heme into HSA. FIG. 10A shows a size exclusion HPLC of HSA and heme-HSA. UV-visible absorbance was monitored at 413 nm, and showed no heme in native HSA, whereas heme-HSA absorbs at 413 nm. FIG. 10B shows size exclusion HPLC of HSA and heme-HSA. Fluorescence emission was monitored at 330 nm (excitation at 285 nm), and showed quenching of the fluorescence emission by heme in heme-HSA, whereas HSA has a strong emission at 330 nm. FIG. 10C shows an x-ray crystal structure of HSA (PDB: 1E78). FIG. 10D shows an x-ray crystal structure of heme-HSA (PDB: 1N5U) (heme molecule appears in red).

FIG. 11 is a synthesis and purification schematic for heme-albumin (heme-HSA). The protein complex was synthesized under basic conditions before adjustment of the pH to 7.4 and buffer exchanged over a 50 kDa TFF hollow fiber filter into PBS pH=7.4.

FIG. 12A shows a size exclusion HPLC (SEC-HPLC) of albumin and heme-albumin monitored via fluorescence spectrometry. Incorporation of heme into albumin was detected by fluorescence quenching at Ex/Em=285/330 (top). Unbound amino acids within HSA fluoresce at the measured wavelengths as shown by the dotted line in the figure. Binding of heme to the protein quenches the produced signal shown by the reduced fluorescent intensity

FIG. 12B shows a CD spectra of heme-albumin and native HSA measured with a Jasco J-815 CD spectrophotometer (Bottom). Unfolding and folding of HSA for formation of the heme-albumin does not alter the secondary structure of the protein complex.

FIG. 13 shoes the hydrodynamic diameter of heme-albumin loaded PDA NPs and unloaded PDA NPs measured with a BI-200SM GONIOMETER. Incorporation of heme-albumin is demonstrated by an increase in hydrodynamic diameter compared to unloaded particles

FIGS. 14A-14D show TEM images of both unloaded (FIG. 14A) and heme-albumin loaded PDA NPs (FIG. 14B), confirming the spherical morphology of the NPs was maintained with the addition of the protein complex. SEM images of unloaded (FIG. 14C) and loaded (FIG. 14D) NPs show the surface coating of heme-albumin.

FIG. 15 shows the in vitro release of heme-albumin from heme-albumin loaded PDA NPs incubated at 1 mg/mL. Increasing oxidative stress with higher concentrations of H₂O₂ increased release rate, demonstrating ROS-responsive release of protein therapeutics from PDA NPs (n=3) Error bars represent the standard deviation.

FIG. 16 shows the cytotoxicity analysis of heme-albumin, PDA NPs, and heme-albumin loaded PDA NPs. ARPE-19 cells were incubated with varying concentrations of therapeutic and cell viability was measured with the MTS assay (n=6). Statistically significant differences for p<0.05 is denoted with *. Heme-albumin showed no significant cytotoxicity to the ARPE-19 cells and both unloaded and heme-albumin loaded PDA NPs only showed significant loss of cell viability at a concentration of 200 μg/mL

FIG. 17 shows the ability of treatment groups to reduce oxidative stress in a LPS model of inflammation (n=6). Statistical significance at p<0.05 is denoted with *. Heme-albumin reduced inflammatory oxidative stress by 22±10% and heme-albumin loaded PDA NPs stress at concentrations above 20 μg/mL (p<0.05). Heme-albumin loaded PDA NPs reduced oxidative stress by 25±10% at concentrations of 100 μg/mL compared to the untreated control.

FIG. 18 shows the ability of treatment groups to reduce oxidative stress in H₂O₂ ROS model (n=6). Significance of p<0.05 is denoted with *. Heme-albumin was able reduce oxidative stress by 100 μM H₂O₂ by 17%. When co-treated with H₂O₂. heme-albumin loaded PDA NPs were able to reduce oxidative stress by 34±6%.

FIG. 19 is a plot showing the HO-1 expression at basal, stressed and co-treatment with 500 μg/mL heme-albumin. Compared to basal and stressed ARPE-19 cells, application with heme-albumin showed a significant increase in HO-1 concentration of almost 3 times the protein expressed. (*p<0.05).

FIG. 20 is a plot showing the phospho-p38MAPK expression at basal, stressed and co-treatment with 500 μg/mL heme-albumin. Compared to basal control, application with heme-albumin showed a significant increase in phospho-p38 MAPK expression (*p<0.05).

FIG. 21 shows PCR quantification of pro-inflammatory cytokines, IL-1β and IL-6. Treatment of ARPE cells with heme-albumin did not induce significant additional pro-inflammatory protein expression compared to control.

FIG. 22 is a plot showing the qtPCR of pro-inflammatory and apoptotic gene expression in oxidative conditions with 500 μg/mL heme-albumin treatment. A statistically significant reduction in caspase-9 gene expression (p=0.0468) was measured.

FIG. 23 is a plot showing the IL-1β expression in ARPE-19 cell lysate (open markers) and secreted media (closed markers) treated with basal, oxidatively challenged, or co-treatment with heme-albumin and oxidative challenged conditions. Treatment with heme-albumin induced a statistically significant (p<0.05) increase in secreted IL-1β, compared to untreated control, relating to the relationship between IL-1β and p38 MAPK activation.

FIG. 24 shows the increased expression of p38 MAPK by co-treatment of 500 μg/mL heme-albumin with 100 μM H₂O₂ compared to treatment with 100 μM H₂O₂ or basal conditions alone shown by immunofluorescence staining. ARPE-19 cells were treated with each treatment group for 24 hours before fixation, permeabilization, and staining with p38 MAPK antibody

### DETAILED DESCRIPTION

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used The materials, methods, and examples are illustrative only and not intended to be limiting. All publications, patent applications, patents, sequences, database entries, and other references mentioned herein are incorporated by reference in their entirety. In case of conflict, the present specification, including definitions, will control

“Active Agent”, as used herein, refers to a physiologically or pharmacologically active substance that acts locally and/or systemically in the body. An active agent is a substance that is administered to a patient for the treatment (e.g., therapeutic agent), prevention (e.g., prophylactic agent), or diagnosis (e.g., diagnostic agent) of a disease or disorder. “Ophthalmic Drug” or “Ophthalmic Active Agent”, as used herein, refers to a therapeutic or prophylactic agent that is administered to a patient to alleviate, delay onset of, or prevent one or more symptoms of a disease or disorder of the eye, or diagnostic agent useful for imaging or otherwise assessing the eye.

“Effective amount” or “therapeutically effective amount”, as used herein, refers to an amount of polymer-drug conjugate effective to alleviate, delay onset of, or prevent one or more symptoms of a disease or disorder being treated by the active agent, and/or an amount of polymer-drug conjugate effective to produce a desired diagnostic signal. In the case of age-related macular degeneration, the effective amount of the polymer-drug conjugate delays, reduces, or prevents vision loss in a patient.

“Biocompatible” and “biologically compatible”, as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient.

“Biodegradable Polymer” as used herein, generally refers to a polymer that will degrade or erode by enzymatic action or hydrolysis under physiologic conditions to smaller units or chemical species that are capable of being metabolized, eliminated, or excreted by the subject. The degradation time is a function of polymer composition, morphology, such as porosity, particle dimensions, and environment.

“Nanoparticle”, as used herein, generally refers to a particle having a diameter, such as an average diameter, from about 10 nm up to but not including about 1 micron, preferably from 100 nm to about 1 micron. The particles can have any shape. Nanoparticles having a spherical shape are generally referred to as “nanospheres”.

“Microparticle”, as used herein, generally refers to a particle having a diameter, such as an average diameter, from about 1 micron to about 100 microns, preferably from about 1 to about 50 microns, more preferably from about 1 to about 30 microns, most preferably from about 1 micron to about 10 microns. The microparticles can have any shape. Microparticles having a spherical shape are generally referred to as “microspheres”.

“Molecular weight” as used herein, generally refers to the relative average chain length of the bulk polymer, unless otherwise specified. In practice, molecular weight can be estimated or characterized using various methods including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions

“Mean particle size” as used herein, generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

“Monodisperse” and “homogeneous size distribution”, are used interchangeably herein and describe a population of nanoparticles or microparticles where all of the particles are the same or nearly the same size. As used herein, a monodisperse distribution refers to particle distributions in which 90% or more of the distribution lies within 15% of the median particle size, more preferably within 10% of the median particle size, most preferably within 5% of the median particle size.

“Pharmaceutically Acceptable”, as used herein, refers to compounds, carriers, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “heme” as used herein refers to a prosthetic group comprising an iron atom in the center of a large organic cyclic macromolecule called porphyrin.

Ranges of values defined herein include all values within the range as well as all sub-ranges within the range. For example, if the range is defined as an integer from 0 to 10, the range encompasses all integers within the range and any and all subranges within the range, e.g., 1-10, 1-6, 2-8, 3-7, 3-9, etc.

### Compositions

Provided herein are ocular therapeutic compositions that comprise a therapeutically effective amount of heme to treat or prevent an ophthalmological disorder in a subject in need thereof.

In some embodiments, the heme can comprise free heme. In other embodiments, the heme can comprise a heme conjugate (e.g., heme covalently or non-covalently associated with a protein or peptide). In some embodiments, the heme conjugate can comprise heme-human serum albumin (heme-HSA). In some embodiments, the heme conjugate can comprise methemoglobin. In some cases, the methemoglobin can be conjugated to HSA. In some embodiments, the heme conjugate can comprise polymerized methemoglobin. In some embodiments, the heme conjugate can comprise a methemoglobin-haptoglobin conjugate.

In some embodiments, the ocular therapeutic composition can further comprise a delivery vehicle for delivery of the heme to the eye. The drug delivery vehicle can comprise, for example, gels (e.g., hydrogels such as an alginate hydrogel, hyaluronic acid hydrogel, or polyethylene glycol based hydrogel), capsules, particles, or other vehicles that modulate solubility, delivery, stability, and or release of the heme within the eye. In some embodiments, the delivery vehicle can comprise a population of particles formed from a biocompatible polymer, and wherein the heme is associated with the particles.

In some embodiments, the heme can be encapsulated within the particles. In some embodiments, the heme can be non-covalently associated with the particles, such as adsorbed to the surface of the particles (or within pores on the surface of the particles).

In some embodiments, the particles can comprise stimuli-responsive particles (e.g., particles which undergo a change in response to a stimulus, such as a change in pH, a change in temperature, or the presence of other species, triggering release of their therapeutic cargo). In certain embodiments, the heme can be released upon exposure of the particles to reactive oxygen species (e.g., the particles are responsive to the presence of ROS).

The biocompatible polymer can comprise any suitable biocompatible polymer. In some embodiments, the biocompatible polymer can comprise a biodegradable polymer. In certain embodiments, the biocompatible polymer can comprise polydopamine.

Polydopamine is formed by the oxidation of dopamine. It is biomimetic of the proteins on the extremity of mussel byssus which are extremely right in L-DOPA and L-Lysine residues. These amino acid residues, containing catechol and amino functional groups, allow for strong adhesion of the mussel to all kinds of substrates in the wet and slightly basic environment of sea water. Because of this, polydopamine has traditionally found extensive use in adhesive coatings.

Methods for forming polydopamine particles are known in the art. Typically, polydopamine particles are formed alkaline aqueous solutions (for example, in the presence of Tris buffer at pH=8.5) in the presence of an oxidant. Oxygen dissolved in the aqueous solution is typically used as the oxidant, but other oxidants may be used, for example ammonium peroxodisulfate or sodium periodate. In some embodiments, the polydopamine particles as used in the present disclosure are essentially spherical, spheroid, ellipsoid, or combinations thereof.

In some embodiments, the particles as used in the present disclosure have an average particle size ranging from about 10 nm to about 1000 nm, for example from about 100 nm to about 1000 nm, from about 200 nm to about 1000 nm, from about 300 nm to about 1000 nm, from about 400 nm to about 1000 nm, from about 500 nm to about 1000 nm, from about 600 nm to about 1000 nm, from about 700 nm to about 1000 nm, from about 800 nm to about 1000 nm, from about 900 nm to about 1000 nm, from about 10 nm to about 900 nm, from about 100 nm to about 900 nm, from about 200 nm to about 900 nm, from about 300 nm to about 900 nm, from about 400 nm to about 900 nm, from about 500 nm to about 900 nm, from about 600 nm to about 900 nm, from about 700 nm to about 900 nm, from about 800 nm to about 900 nm, from about 10 nm to about 800 nm, from about 100 nm to about 800 nm, from about 200 nm to about 800 nm, from about 300 nm to about 800 nm, from about 400 nm to about 800 nm, from about 500 nm to about 800 nm, from about 600 nm to about 800 nm, from about 700 nm to about 800 nm, from about 10 nm to about 700 nm, from about 100 nm to about 700 nm, from about 200 nm to about 700 nm, from about 300 nm to about 700 nm, from about 400 nm to about 700 nm, from about 500 nm to about 700 nm, from about 600 nm to about 700 nm, from about 10 nm to about 600 nm, from about 100 nm to about 600 nm, from about 200 nm to about 600 nm, from about 300 nm to about 600 nm, from about 400 nm to about 600 nm, from about 500 nm to about 600 nm, from about 10 nm to about 500 nm, from about 100 nm to about 500 nm, from about 200 nm to about 500 nm, from about 300 nm to about 500 nm, from about 400 nm to about 500 nm, from about 10 nm to about 400 nm, from about 100 nm to about 400 nm, from about 200 nm to about 400 nm, from about 300 nm to about 400 nm, from about 10 nm to about 300 nm, from about 100 nm to about 300 nm, from about 200 nm to about 300 nm, from about 10 nm to about 200 nm, from about 100 nm to about 200 nm, or from about 10 nm to about 100 nm. In certain embodiments, the population of particles can have an average particle size of from about 10 nm to about 1000 nm, such as from about 100 nm to about 400 nm, from about 100 nm to about 200 nm, from about 120 nm to about 270 nm, or from about 120 nm to about 170 nm.

In some embodiments, the population of particles has an average particle size of about 10 nm, about 25 nm, about 50 nm, about 75 nm, about 100 nm, about 125 nm, about 150 nm, about 175 nm, about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm.

In some embodiments, the particles as described herein may be further comprise a coating. The coating can be disposed on the surface of the particle, for example by bonding, adsorption or by complexation. The coating can also be intermingled or dispersed within the particle as well as disposed on the surface of the particle.

In some embodiments, the coating may comprise a coating polymer, i.e., the particles as described herein may be coated with a polymer. In some embodiments, the polymer may comprise an alginate. In some embodiments, the polymer may comprise polyethylene glycol, polyvinyl alcohol, or similar substances. In some embodiments, the coating may also comprise a non-ionic surfactant such as those composed of polyalkylene oxide, e.g. polyethylene glycol or polypropylene glycol, and can include a copolymer of more than one alkylene oxide. In some embodiments, the coating can comprise a polyoxyethylene-polyoxypropylene copolymer, i.e., a poloxamer such a poloxamer 188, 237, 338, and 407. In certain embodiments, the particles can be further coated with a coating polymer such as alginate, a polyalkylene oxide (e.g., PEG), a polyester (e.g., polylactic acid, polyglycolic acid, PLGA, polycaprolactone), polyvinyl alcohol, copolymers thereof, and combinations thereof.

In some embodiments, the particles have a loading efficiency of the therapeutic of greater than 0.1%, for example greater than 0.5%, greater than 1%, greater than 5%, greater than 10%, greater than 15%, greater than 20%, greater than 25%, greater than 30%, greater than 35%, greater than 40%, greater than 45%, or greater than 50%. In some embodiments, the therapeutic agent (for example, the heme) may be loaded in the particles in an amount of about 10 μg per mg, about 20 μg per mg, about 30 μg per mg, about 40 μg per mg, about 50 μg per mg, about 60 μg per mg, about 70 μg per mg, about 80 μg per mg, about 90 μg per mg, about 100 μg per mg, about 110 μg per mg, about 120 μg per mg, about 130 μg per mg, about 140 μg per mg, about 150 μg per mg, about 160 μg per mg, about 170 μg per mg, about 180 μg per mg, about 190 μg per mg, about 200 μg per mg, about 225 μg per mg, about 250 μg per mg, about 275 μg per mg, about 300 μg per mg, about 325 μg per mg, about 350 μg per mg, about 375 μg per mg, about 400 μg per mg, about 425 μg per mg, about 450 μg per mg, about 475 μg per mg, about 500 μg per mg, or more

In some embodiments, the composition can further comprise an additional active agent (in addition to the heme). The additional active agent can also be incorporated in the particles. In some embodiments, the additional active agent can be encapsulated within the particles. In some embodiments, the additional active agent can be non-covalently associated with the particles, such as adsorbed to the surface of the particles (or within pores on the surface of the particles).

The additional active agent can comprise a therapeutic, diagnostic, and/or prophylactic agent. The active agent can be a small molecule active agent and/or a biomolecule, such as an enzyme, protein, antibody, growth factor, polypeptide, polysaccharide, lipid, or nucleic acid. Suitable small molecule active agents include organic and organometallic compounds. In some instances, the small molecule active agent has a molecular weight of less than about 2000 g/mol, preferably less than about 1500 g/mol, more preferably less than about 1200 g/mol, most preferably less than about 1000 g/mol. In other embodiments, the small molecule active agent has a molecular weight less than about 500 g/mol. The small molecule active agent can be a hydrophilic, hydrophobic, or amphiphilic compound. Biomolecules typically have a molecular weight of greater than about 2000 g/mol and may be composed of repeat units such as amino acids (peptide, proteins, enzymes, etc.) or nitrogenous base units (nucleic acids).

In certain embodiments, the additional active agent is an ophthalmic drug. In particular embodiments, the additional active agent is a drug used to treat, prevent or diagnose a disease or disorder of the posterior segment eye. Non-limiting examples of ophthalmic drugs include anti-glaucoma agents, anti-angiogenesis agents, anti-infective agents, anti-inflammatory agents, growth factors, immunosuppressant agents, anti-allergic agents, complement inhibitors, and combinations thereof.

Representative anti-glaucoma agents include prostaglandin analogs (such as travoprost, bimatoprost, and latanoprost), beta-andrenergic receptor antagonists (such as timolol, betaxolol, levobetaxolol, and carteolol), alpha-2 adrenergic receptor agonists (such as brimonidine and apraclonidine), carbonic anhydrase inhibitors (such as brinzolamide, acetazolamine, and dorzolamide), miotics (i.e., parasympathomimetics, such as pilocarpine and ecothiopate), seretonergics muscarinics, dopaminergic agonists, and adrenergic agonists (such as apraclonidine and brimonidine).

Representative anti-angiogenesis agents include, but are not limited to, antibodies to vascular endothelial growth factor (VEGF) such as bevacizumab (AVASTIN®) and rhuFAb V2 (ranibizumab, LUCENTIS®), and other anti-VEGF compounds such as aflibercept (EYLEA®), faricimab, and brolucizumab; MACUGEN® (pegaptanim sodium, anti-VEGF aptamer or EYE001) (Eyetech Pharmaceuticals); pigment epithelium derived factor(s) (PEDF), COX-2 inhibitors such as celecoxib (CELEBREX®) and rofecoxib (VIOXX®); interferon alpha; interleukin-12 (IL-12); thalidomide (THALOMID®) and derivatives thereof such as lenalidomide (REVLIMID®), squalamine; endostatin; angiostatin; ribozyme inhibitors such as ANGIOZYME® (Sirna Therapeutics); multifunctional antiangiogenic agents such as NEOVASTAT® (AE-941) (Aeterna Laboratories, Quebec City, Canada); receptor tyrosine kinase (RTK) inhibitors such as sunitinib (SUTENT®); tyrosine kinase inhibitors such as sorafenib (Nexavar®) and erlotinib (Tarceva®); antibodies to the epidermal grown factor receptor such as panitumumab (VECTIBIX®) and cetuximab (ERBITUX®); as well as other anti-angiogenesis agents known in the art.

Anti-infective agents include antiviral agents, antibacterial agents, antiparasitic agents, and anti-fungal agents. Representative antiviral agents include ganciclovir and acyclovir. Representative antibiotic agents include aminoglycosides such as streptomycin, amikacin, gentamicin, and tobramycin, ansamycins such as geldanamycin and herbimycin, carbacephems, carbapenems, cephalosporins, glycopeptides such as vancomycin, teicoplanin, and telavancin, lincosamides, lipopeptides such as daptomycin, macrolides such as azithromycin, clarithromycin, dirithromycin, and erythromycin, monobactams, nitrofurans, penicillins, polypeptides such as bacitracin, colistin and polymyxin B, quinolones, sulfonamides, polyhexamethylene biguanide (PHMB), and tetracyclines.

In some cases, the active agent is an anti-allergic agent such as olopatadine and epinastine.

Anti-inflammatory agents include both non-steroidal and steroidal anti-inflammatory agents. Suitable steroidal active agents include glucocorticoids, progestins, mineralocorticoids, and corticosteroids.

A variety of complement inhibitors are known in the art, and include, for example, C1-Inh (Cetor/Sanquin, BerinertP/CSL Behring, Lev Pharma), Rhucin/rhCl1NH (Pharming Group N.V.), sCR1/TP10 (Avant Immunotherpeutics), CAM-2/MLN-2222 (Millenium Pharmaceuticals), eculuizumab/soliris (Alexion Pharmaceuticals), Pexelizumab (Alexion Pharmaceuticals), Ofatumumab (Genmab A/S), APL-2 (Apellis Pharmaceuticals), sDAF, sMCP, sMCP-DAF, sCDS9, DAF-cd59, C5a mutants, Antii-0S, Anti-C3, Anti-C3a, Anti-05a, NMeFKPdChaWdR, F-(OpdChaWR), Compastatin/POT-4 (Potentia Pharmaceuticals), OMS721 (Omeros Corporation), AMY-101 (Amyndas Pharmaceuticals SA), PMX-53 (Peptech Ltd.), rhMBL (Enzon Pharmaceuticals), the anti-C5 aptamer avacincaptad pegol/ARC1905 (Zimura®; IVERIC Bio [formerly, Ophthotech], Cranbury, NJ, USA), BCX-1470, FUT-175, K-76, thioester inhibitors, and factor D inhibitors such as ACH-4471 (Achillion Pharmaceuticals, Inc. (New Haven, Conn.)).

The ophthalmic drug may be present in its neutral form, or in the form of a pharmaceutically acceptable salt. In some cases, it may be desirable to prepare a formulation containing a salt of an active agent due to one or more of the salt's advantageous physical properties, such as enhanced stability or a desirable solubility or dissolution profile.

Generally, pharmaceutically acceptable salts can be prepared by reaction of the free acid or base forms of an active agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, Md., 2000, p. 704. Examples of ophthalmic drugs sometimes administered in the form of a pharmaceutically acceptable salt include timolol maleate, brimonidine tartrate, and sodium diclofenac.

In some cases, the active agent is a diagnostic agent imaging or otherwise assessing the eye. Exemplary diagnostic agents include paramagnetic molecules, fluorescent compounds, magnetic molecules, and radionuclides, x-ray imaging agents, and contrast media.

The ocular therapeutic compositions described herein may be prepared using a physiological saline solution as a vehicle. The pH of the ocular therapeutic composition may be maintained at a substantially neutral pH (for example, about 7.4, in the range of about 6.5 to about 7.4, etc.) with an appropriate buffer system as known to one skilled in the art (for example, acetate buffers, citrate buffers, phosphate buffers, borate buffers).

Any diluent used in the preparation of the ocular therapeutic compositions may preferably be selected so as not to unduly affect the biological activity of the composition. Example of such diluents which are especially for injectable ophthalmic compositions are water, various saline, organic, or inorganic salt solutions, Ringer's solution, dextrose solution, and Hank's solution.

In addition, the ocular therapeutic compositions may include additives such other buffers, diluents, carriers, adjuvants, or excipients. Any pharmaceutically acceptable buffer suitable for application to the eye may be used, e.g., tris or phosphate buffers. Other agent may be employed in the formulation for a variety of purposes. For example, buffering agents, preservatives, co-solvents, surfactants, oils, humectants, emollients, chelating agents, stabilizers or antioxidants may be employed. Water soluble preservatives which may be employed include, but are not limited to, benzalkonium chloride, chlorobutanol, thimerosal, sodium bisulfate, phenylmercuric acetate, phenylmercuric nitrate, ethyl alcohol, methylparaben, polyvinyl alcohol, benzyl alcohol and phenylethyl alcohol. A surfactant may be Tween 80.

Other vehicles that may be used include, but are not limited to, polyvinyl alcohol, povidone, hydroxypropyl methyl cellulose, poloxamers, carboxymethyl cellulose, hydroxyethyl cellulose, or purified water. Tonicity adjustors may be included, for example, sodium chloride, potassium chloride, mannitol, or glycerin Antioxidants include, but are not limited to, sodium metabisulfite, sodium thiosulfate, acetylcysteine, butylated hydroxyanisole, or butylated hydroxytoluene. The indications, effective doses, formulations, contraindications, etc. of the components in the ophthalmic composition are available and are known to one skilled in the art.

These agents may be present in individual amounts from about 0.001% to about 5% by weight and preferably about 0.01% to about 2% by weight in the formulation. Suitable water soluble buffering agents that may be employed are sodium carbonate, sodium borate, sodium phosphate, sodium acetate, or sodium bicarbonate, as approved by the U.S. FDA for the desired route of administration. These agents may be present in amounts sufficient to maintain a pH of the system between about 2 to about 9 and preferably about 4 to about 8. As such, the buffering agent may be as much as about 5% (w/w) of the total ocular therapeutic composition. Electrolytes such as, but limited to, sodium chloride and potassium chloride may be also included in the formulation.

In some embodiments, the ocular therapeutic composition further comprises a hydrogel. In some embodiments, the hydrogel comprises a polymer composition, for example a homopolymer, a copolymer, or combinations thereof. In some embodiments, the hydrogel comprises one or more hydrophilic polymers, i.e., a polymer having at least 0.1 wt. % solubility in water, for example having at least 0.5 wt. % solubility. In some embodiments, the hydrophilic polymer has a solubility of at least 1 mg/mL.

In some embodiments, the polymer composition may comprise one or more vinyl alcohol residues. In some embodiments, the polymer composition may comprise one or more acrylamide residues In some embodiments, the polymer composition may comprise one or more residues selected from a polyethylene glycol derivative or a functionalized polyethylene glycol. In some embodiments, the polymer composition may comprise one or more acrylate residues or one or more methacrylate residues. In some embodiments, the polymer composition may comprise one or more residues selected from acrylamide, N-ornithine acrylamide, N-(2-hydroxypropyl) acrylamide, hydroxyethylacrylate, hydroxyethylmethacrylate, polyethyleneglycol acrylates, polyethylenegylcol methacrylates, N-vinylpyrrolidinone, N-phenylacrylamide, dimethylaminopropyl methacrylamide, acrylic acid, benzylmethacrylamide, methylthioethylacrulamide, or combinations thereof.

Representative examples of hydrogels which can be used include, but are not limited to, hyaluronic acid, collagen, gellan, silk, fibrin, alginate, chitosan, polyacrylamides and methacrylate derivatives thereof, polyacrylic acid and methacrylate derivatives thereof, polyvinyl alcohol, polyethylene glycol and derivatives thereof, polypropylene glycol and derivatives thereof, or combinations thereof.

In some embodiments, the hydrogel comprises a hyaluronate derivative, for example poly (N-isopropylacrylamide) grafted sodium hyaluronate.

### Methods of Use

The term “administering” or “administration” of a disclosed therapeutic composition to a subject includes any route of introducing or delivering to a subject the device to perform its intended function. Administration can be carried out by any suitable route, including orally, intranasally, parenterally (intravenously, intramuscularly, intraperitoneally, or subcutaneously), or topically. Administration includes self-administration and the administration by another. In some instances, administration is via injection to the eye, including intraocular injection.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

The term “separate” administration refers to an administration of at least two active ingredients at the same time or substantially the same time by different routes.

The term “sequential” administration refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. The term “sequential” therefore is different than “simultaneous” administration.

The term “simultaneous” administration refers to the administration of at least two active ingredients by the same route at the same time or at substantially the same time.

The term “therapeutic” as used herein means a treatment and/or prophylaxis. A therapeutic effect is obtained by suppression, remission, or eradication of a disease state.

The present disclosure further provides methods of treating an ophthalmological disease or disorder by administering a therapeutically effective amount of the ocular therapeutic compositions described herein. In some embodiments, the disclosed methods pertain to treatment of an ophthalmological disorder comprising injecting a therapeutically effective amount of the disclosed ocular therapeutic composition into the eye of a subject. The subject can be a patient; and the patient can have been diagnosed with an ophthalmological disorder. In some aspects, the method can further comprise diagnosing a subject with an ophthalmological disorder.

Also provided are methods of treating an ophthalmological disorder in a subject in need thereof comprising injecting into the eye of the subject a therapeutically effective amount of an ocular therapeutic composition comprising heme (which may be associated with particles as described above, or not associated with particles). As discuss above, in some embodiments, the heme can comprise free heme. In other embodiments, the heme can comprise a heme conjugate (e.g., heme covalently or non-covalently associated with a protein or peptide). In some embodiments, the heme conjugate can comprise heme-HSA. In some embodiments, the heme conjugate can comprise methemoglobin. In some cases, the methemoglobin can be conjugated to HSA. In some embodiments, the heme conjugate can comprise polymerized methemoglobin.

The ophthalmological disorder can be acute macular neuroretinopathy; Behcet's disease; neovascularization, including choroidal neovascularization; diabetic uveitis; histoplasmosis; infections, such as fungal or viral-caused infections; macular degeneration, such as acute macular degeneration (AMD), including wet AMD, dry AMD, non-exudative AMD and exudative AMD; retinal degenerative diseases such as GA, edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic ophthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, a cancer, and glaucoma. In certain instances, the ophthalmological disorder is wet age-related macular degeneration (wet AMD), a cancer, neovascularization, macular edema, or edema.

In certain embodiments, the ophthalmological disorder can comprise an inflammation-mediated disorder. “Inflammation-mediated” in relation to an ocular condition means any condition of the eye which can benefit from treatment with an anti-inflammatory agent, and is meant to include, but is not limited to, uveitis, macular edema, acute macular degeneration, retinal detachment, ocular tumors, fungal or viral infections, multifocal choroiditis, diabetic retinopathy, uveitis, proliferative vitreoretinopathy (PVR), sympathetic ophthalmia, Vogt-Koyanagi-Harada (VKH) syndrome, histoplasmosis, and uveal diffusion.

In a further particular aspect, the ophthalmological disorder is wet age-related macular degeneration (wet AMD). In a further particular aspect, the ophthalmological disorder is dry age-related macular degeneration (dry AMD).

In a further aspect, the ophthalmological disorder can comprise a retinal degenerative disease, such as PCR or geographic atrophy.

In various aspects, the injection for treatment of an ophthalmological disorder can be injection to the vitreous chamber of the eye. In some cases, the injection is an intravitreal injection, a subconjunctival injection, a subtenon injection, a retrobulbar injection, or a suprachoroidal injection.

“Ocular region” or “ocular site” means any area of the ocular globe (eyeball), including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of areas of the eyeball in an ocular region include, but are not limited to, the anterior chamber, the posterior chamber, the vitreous cavity, the choroid, the suprachoroidal space, the conjunctiva, the subconjunctival space, the episcleral space, the intracorneal space, the subretinal space, sub-Tenon's space, the epicorneal space, the sclera, the pars plana, surgically-induced avascular regions, the macula, and the retina.

“Ophthalmological disorder” can mean a disease, ailment or condition which affects or involves the eye or one of the parts or regions of the eye. Broadly speaking, the eye includes the eyeball, including the cornea, and other tissues and fluids which constitute the eyeball, the periocular muscles (such as the oblique and rectus muscles) and the portion of the optic nerve which is within or adjacent to the eyeball.

“Glaucoma” means primary, secondary and/or congenital glaucoma. Primary glaucoma can include open angle and closed angle glaucoma. Secondary glaucoma can occur as a complication of a variety of other conditions, such as injury, inflammation, pigment dispersion, vascular disease and diabetes. The increased pressure of glaucoma causes blindness because it damages the optic nerve where it enters the eye. Thus, in one nonlimiting embodiment, by lowering reactive oxygen species, STC-1, or MSCs which express increased amounts of STC-1, may be employed in the treatment of glaucoma and prevent or delay the onset of blindness.

“Injury” or “damage” in relation to an ocular condition are interchangeable and refer to the cellular and morphological manifestations and symptoms resulting from an inflammatory-mediated condition, such as, for example, inflammation, as well as tissue injuries caused by means other than inflammation, such as chemical injury, including chemical burns, as well as injuries caused by infections, including but not limited to, bacterial, viral, or fungal infections

“Intraocular” means within or under an ocular tissue. An intraocular administration of an ocular therapeutic composition includes administration of the ocular therapeutic composition to a sub-tenon, subconjunctival, suprachoroidal, subretinal, intravitreal, anterior chamber, and the like location. An intraocular administration of an ocular therapeutic composition excludes administration of the drug delivery system to a topical, systemic, intramuscular, subcutaneous, intraperitoneal, and the like location.

“Macular degeneration” refers to any of a number of disorders and conditions in which the macula degenerates or loses functional activity. The degeneration or loss of functional activity can arise as a result of, for example, cell death, decreased cell proliferation, loss of normal biological function, or a combination of the foregoing. Macular degeneration can lead to and/or manifest as alterations in the structural integrity of the cells and/or extracellular matrix of the macula, alteration in normal cellular and/or extracellular matrix architecture, and/or the loss of function of macular cells. The cells can be any cell type normally present in or near the macula including RPE cells, photoreceptors, and capillary endothelial cells. Age-related macular degeneration, or AMD, is the major macular degeneration related condition, but a number of others are known including, but not limited to, Best macular dystrophy, Stargardt macular dystrophy, Sorsby fundus dystrophy, Mallatia Leventinese, Doyne honeycomb retinal dystrophy, and RPE pattern dystrophies Age-related macular degeneration is described as either “dry” or “wet.” The wet, exudative, neovascular form of AMD affects about 10-20% of those with AMD and is characterized by abnormal blood vessels growing under or through the retinal pigment epithelium (RPE), resulting in hemorrhage, exudation, scarring, or serous retinal detachment. Eighty to ninety percent of AMD patients have the dry form characterized by atrophy of the retinal pigment epithelium and loss of macular photoreceptors. Drusen may or may not be present in the macula. There may also be geographic atrophy of retinal pigment epithelium in the macula accounting for vision loss. At present there is no cure for any form of AMD, although some success in attenuation of wet AMD has been obtained with photodynamic and especially anti-VEGF therapy and complement inhibition.

“Drusen” is debris-like material that accumulates with age below the RPE. Drusen is observed using a funduscopic eye examination. Normal eyes may have maculas free of drusen, yet drusen may be abundant in the retinal periphery. The presence of soft drusen in the macula, in the absence of any loss of macular vision, is considered an early stage of AMD. Drusen contains a variety of lipids, polysaccharides, and glycosaminoglycans along with several proteins, modified proteins or protein adducts. There is no generally accepted therapeutic method that addresses drusen formation and thereby manages the progressive nature of AMD.

“Ocular neovascularization” (ONV) is used herein to refer to choroidal neovascularization or retinal neovascularization, or both.

“Retinal neovascularization” (RNV) refers to the abnormal development, proliferation, and/or growth of retinal blood vessels, e.g., on the retinal surface.

“Subretinal neovascularization” (SRNVM) refers to the abnormal development, proliferation, and/or growth of blood vessels beneath the surface of the retina.

“Cornea” refers to the transparent structure forming the anterior part of the fibrous tunic of the eye It consists of five layers, specifically: 1) anterior corneal epithelium, continuous with the conjunctiva; 2) anterior limiting layer (Bowman's layer); 3) substantia propria, or stromal layer: 4) posterior limiting layer (Descemet's membrane); and 5) endothelium of the anterior chamber or keratoderma.

“Retina” refers to the innermost layer of the ocular globe surrounding the vitreous body and continuous posteriorly with the optic nerve. The retina is composed of layers including the: 1) internal limiting membrane; 2) nerve fiber layer; 3) layer of ganglion cells; 4) inner plexiform layer; 5) inner nuclear layer; 6) outer plexiform layer; 7) outer nuclear layer; 8) external limiting membrane; and 9) a layer of rods and cones.

“Retinal degeneration” refers to any hereditary or acquired degeneration of the retina and/or retinal pigment epithelium. Non-limiting examples include retinitis pigmentosa, Best's Disease, RPE pattern dystrophies, and age-related macular degeneration.

In various aspects, a method of treating an ophthalmological disorder may comprise treatment of various ocular diseases or conditions of the retina, including the following: maculopathies/retinal degeneration: macular degeneration, including age-related macular degeneration (AMD), such as non-exudative age-related macular degeneration and exudative age-related macular degeneration; choroidal neovascularization; retinopathy, including diabetic retinopathy, acute and chronic macular neuroretinopathy, central serous chorioretinopathy; and macular edema, including cystoid macular edema, and diabetic macular edema. Uveitis/retinitis/choroiditis: acute multifocal placoid pigment epitheliopathy, Behcet's disease, birdshot retinochoroidopathy, infectious (syphilis, Lyme Disease, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome. Vascular diseases/exudative diseases: retinal arterial occlusive disease, central retinal vein occlusion, disseminated intravascular coagulopathy, branch retinal vein occlusion, hypertensive fundus changes, ocular ischemic syndrome, retinal arterial microaneurysms, Coats disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease, Traumatic/surgical diseases. sympathetic ophthalmia, uveitic retinal disease, retinal detachment, trauma, laser, PDT, photocoagulation, hypoperfusion during surgery, radiation retinopathy, bone marrow transplant retinopathy. Proliferative disorders: proliferative vitreal retinopathy and epiretinal membranes, proliferative diabetic retinopathy. Infectious disorders: ocular histoplasmosis, ocular toxocariasis, ocular histoplasmosis syndrome (OHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis. Genetic disorders: retinitis pigmentosa, systemic disorders with associated retinal dystrophies, congenital stationary night blindness, cone dystrophies, Stargardt's disease and fundus flavimaculatus, Best's disease, pattern dystrophy of the retinal pigment epithelium, X-linked retinoschisis, Sorsby's fundus dystrophy, benign concentric maculopathy, Bietti's crystalline dystrophy, pseudoxanthoma elasticum. Retinal tears/holes: retinal detachment, macular hole, giant retinal tear. Tumors: retinal disease associated with tumors, congenital hypertrophy of the RPE, posterior uveal melanoma, choroidal hemangioma, choroidal osteoma, choroidal metastasis, combined hamartoma of the retina and retinal pigment epithelium, retinoblastoma, vasoproliferative tumors of the ocular fundus, retinal astrocytoma, and intraocular lymphoid tumors. Miscellaneous: punctate inner choroidopathy, acute posterior multifocal placoid pigment epitheliopathy, myopic retinal degeneration, acute retinal pigment epithelitis and the like.

An anterior ocular condition is a disease, ailment or condition which affects or which involves an anterior (i.e., front of the eye) ocular region or site, such as a periocular muscle, an eyelid or an eyeball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the iris but in front of the posterior wall of the lens capsule), the lens or the lens capsule and blood vessels and nerve which vascularize or innervate an anterior ocular region or site.

Thus, an anterior ocular condition can include a disease, ailment or condition, such as for example, aphakia; pseudophakia; astigmatism; blepharospasm; cataract; posterior capsule opacification (PCO); conjunctival diseases; conjunctivitis, including, but not limited to, atopic keratoconjunctivitis; corneal injuries, including, but not limited to, injury to the corneal stromal areas; corneal diseases; corneal ulcer; dry eye syndromes; eyelid diseases; lacrimal apparatus diseases; lacrimal duct obstruction; myopia; presbyopia; pupil disorders, refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

Other diseases or disorders of the eye which may be treated in accordance with the present invention include, but are not limited to, ocular cicatricial pemphigoid (OCP), Stevens Johnson syndrome and cataracts.

A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e., the optic disc), and blood vessels and nerves which vascularize or innervate a posterior ocular region or site. Thus, a posterior ocular condition can include a disease, ailment or condition, such as for example, acute macular neuroretinopathy; Behcet's disease; choroidal neovascularization; diabetic retinopathy; uveitis; ocular histoplasmosis; infections, such as fungal or viral-caused infections; macular degeneration, such as acute macular degeneration, non-exudative age-related macular degeneration and exudative age-related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial or venous occlusive disease, retinal detachment, uveitic retinal disease; sympathetic ophthalmia; Vogt-Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal ganglion cells or retinal nerve fibers (i.e., neuroprotection).

In some embodiments, the ophthalmic disorder is ocular inflammation resulting from, e.g., iritis, conjunctivitis, seasonal allergic conjunctivitis, acute and chronic endophthalmitis, anterior uveitis, uveitis associated with systemic diseases, posterior segment uveitis, chorioretinitis, pars planitis, masquerade syndromes including ocular lymphoma, pemphigoid, scleritis, keratitis, severe ocular allergy, corneal abrasion and blood-aqueous barrier disruption. In yet another embodiment, the ophthalmic disorder is post-operative ocular inflammation resulting from, for example, photorefractive keratectomy, cataract removal surgery, intraocular lens implantation, vitrectomy, corneal transplantation, forms of lamellar keratectomy (DSEK, etc.), and radial keratotomy.

In various aspects, the injection for treatment of an ophthalmological disorder can be injection to the vitreous chamber of the eye. In some cases, the injection is an intravitreal injection, a subconjunctival injection, a subtenon injection, a retrobulbar injection, or a suprachoroidal injection.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1: Therapeutic and Delivery System to Treat Inflammation in Retinal Degenerative Diseases

### Overview

There is strong evidence that inflammation and reactive oxygen species (ROS) play a key role in the pathogenesis of dry AMD (dAMD) and inherited retinal degenerative diseases. Diminishing inflammation and mitigating ROS within the retina could be a game-changing therapeutic approach for these patients who currently lack treatments. Described herein are ocular drug delivery systems, particularly for AMD, including ROS-responsive nanoparticles. These therapeutic strategies can mitigate oxidative injury. These strategies involve administration of therapeutic heme bound to human serum albumin (heme-albumin) exhibits anti-inflammatory effects systemically due to its degradation components. This therapeutic targets pathways that have been directly implicated in the pathogenesis of dAMD and retinopathies including retinitis pigmentosa. Therefore, it follows that heme-albumin has the potential to treat the inflammatory aspects of dAMD and inherited retinal degeneration. Our pan-disease therapeutic approach is likely to succeed since we are harnessing the healing response of the body with naturally derived therapeutics and drug delivery systems to treat retinal inflammation. This has significant translational potential for multiple retinal degenerative diseases, including dAMD.

### Introduction

Inflammation and ROS play a key role in the pathogenesis of retinal degenerative diseases. Blood samples and drusen of dAMD patients have higher levels of inflammatory proteins, and studies have linked the complement system with elevated risk of developing dAMD. Antioxidant supplementation is the only known intervention that has been shown to reduce progression of dAMD by mitigating ROS, but has several drawbacks including high dosing to overcome the blood-retinal barrier. Thus, diminishing inflammation and ROS locally at the retina has potential to treat multiple retinal degenerative diseases, including dAMD and retinitis pigmentosa.

Heme bound to human serum albumin (heme-albumin) exhibits anti-inflammatory effects systemically due to its degradation components, particularly carbon monoxide and biliverdin. To overcome the hydrophobicity and low molecular weight of heme, we exploit the specific binding of heme to human serum albumin (HSA). The carrier protein HSA can be used to store and transport heme to retinal cells. HSA contains multiple hydrophobic molecule binding pockets, naturally serves as a transporter, and has inherent antioxidant properties. Therefore, HSA is an ideal carrier protein for heme. Further, heme-albumin is proposed as a therapeutic for dAMD because the accumulation of hemoglobin-scavenging CD163(+) macrophages have been recently discovered in dAMD. Since these retinal diseases are associated with inflammation, it is proposed that delivery of heme to the eye should reduce inflammation via HO-1 catalyzed production of carbon monoxide. The therapeutic can be delivered with polydopamine nanoparticles because this naturally derived system releases more therapeutic in response to ROS, sustains release while preserving bioactivity, and has anti-inflammatory degradation products.

### Strategy for Treatment

Our goal is to develop a pan-disease treatment for retinal degenerative diseases. We propose to locally sustain release of heme-albumin from a ROS-responsive nanoparticle delivery system to extend release and protect therapeutic bioactivity while mitigating retinal inflammation and degeneration. We hypothesize that heme-albumin will induce retinal cells to overexpress HO-1, reducing inflammatory markers. We further hypothesize that the ROS-responsive nanoparticle delivery system will sustain release and preserve anti-inflammatory bioactivity of heme-albumin for 6 months.

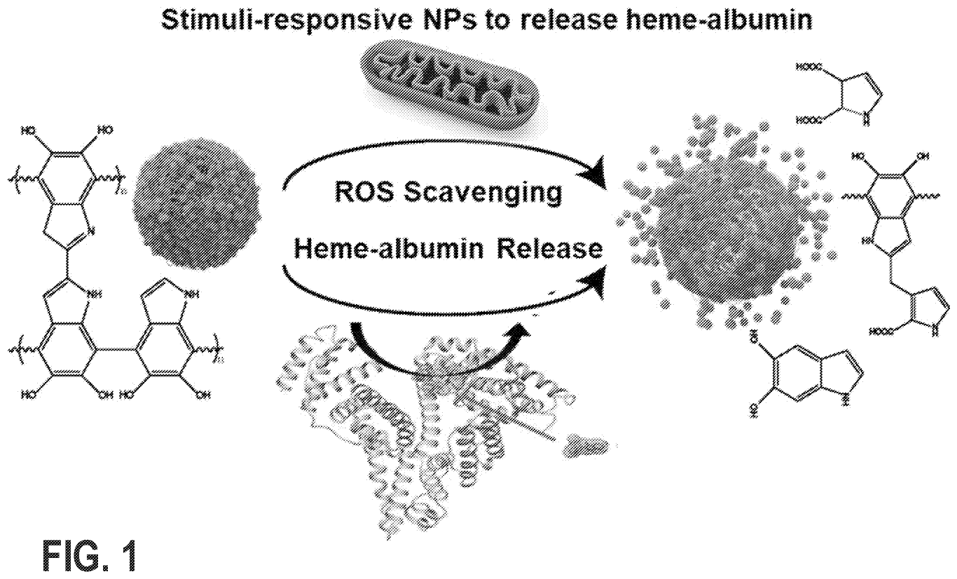

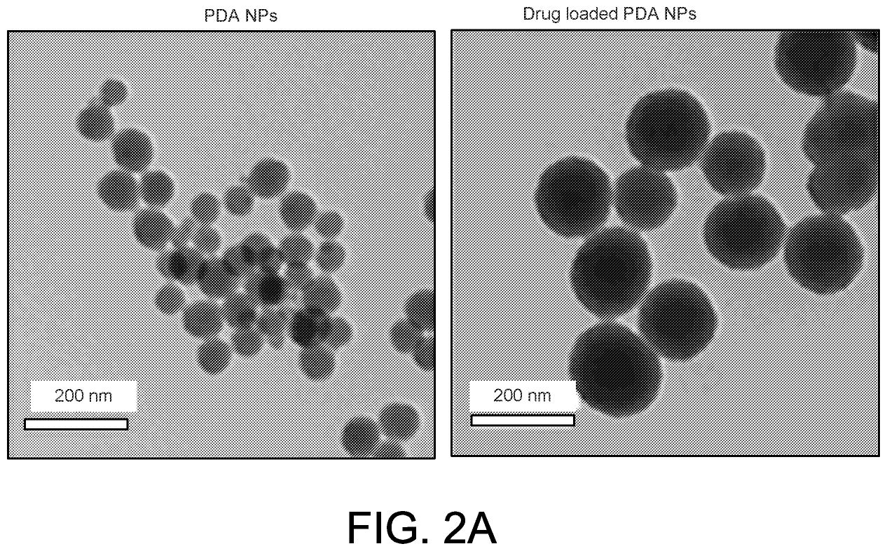

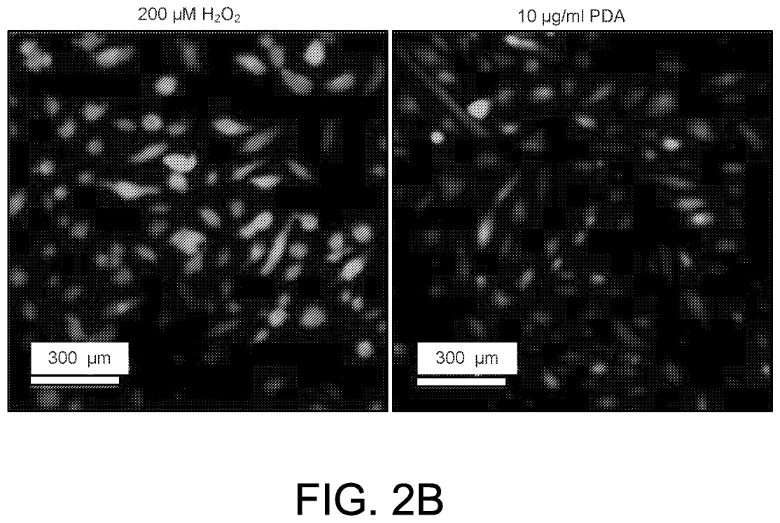

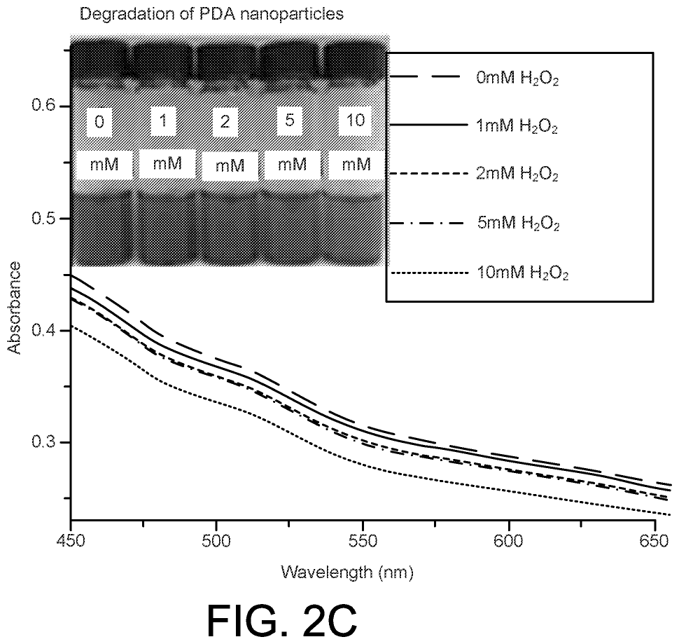

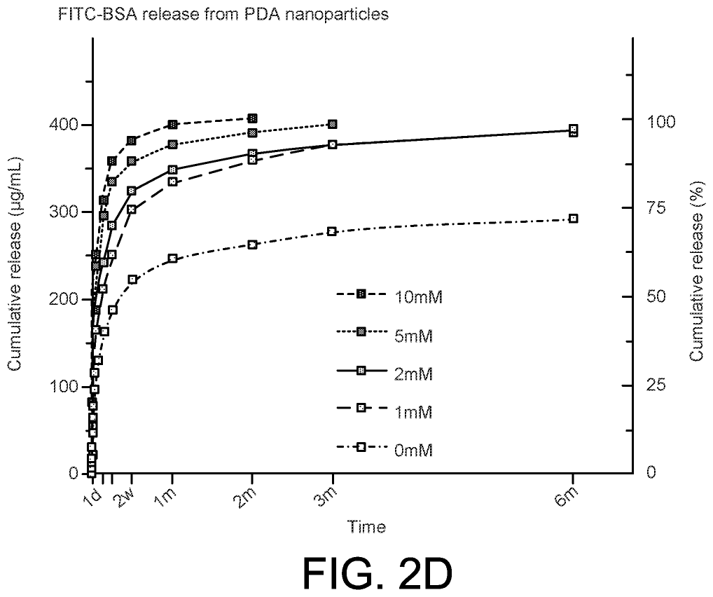

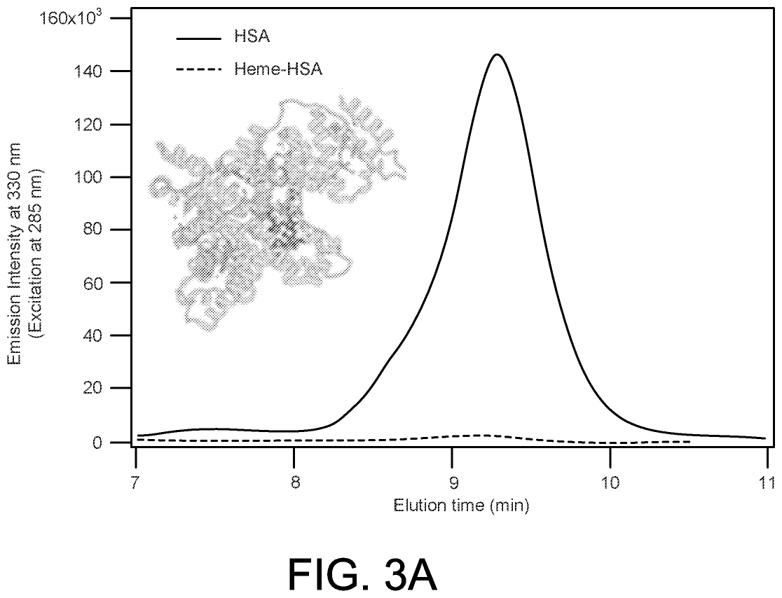

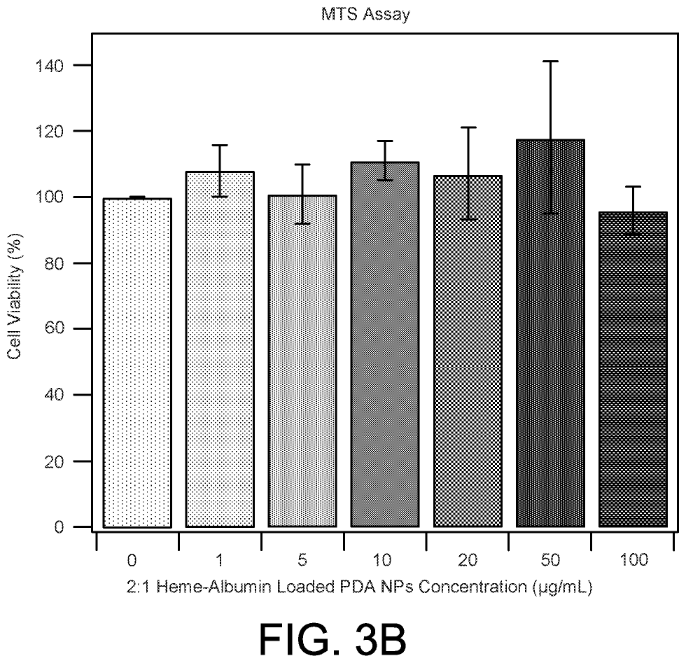

We propose a ROS-scavenging polydopamine (PDA) nanoparticle delivery system to sustain release of anti-inflammatory heme-albumin (FIG. 1). The building block of the nanoparticles, dopamine, is prevalent in the human body and can inhibit inflammation by scavenging ROS. Moreover, the cavities on the nanoparticle surfaces enable therapeutic loading, and bioactivity is preserved by gentle loading chemistry. As a proof of concept, PDA nanoparticles were synthesized and loaded with model protein bovine serum albumin (BSA), which bas similar molecular weight as heme-albumin. Nanoparticles were 160 nm in diameter (FIG. 2A), enabling injection through a small gauge needle. These particles efficiently scavenged ROS in vitro using an ARPE-19 cell culture model (FIG. 2B) PDA degraded more rapidly in the presence of high levels of ROS (FIG. 2C). The reaction of PDA nanoparticles with ROS also triggered the release of therapeutics, with sustained release for up to 6 months (FIG. 2D). We have incorporated heme into HSA, as shown by HPLC (FIG. 3A). FIG. 3B shows preliminary data demonstrating biocompatibility of PDA nanoparticles loaded with heme-albumin.

The delivery of anti-inflammatory therapeutics to the eye is a reasonable strategy to resolve inflammation in dry AMD to prevent progression to wet AMD and permanent blindness. Fortunately, the hydrophobic molecule heme can be metabolized in cells by the enzyme HO-1 to yield biliverdin and carbon monoxide, which have anti-inflammatory effects. Since heme is not soluble in aqueous media, heme-containing protein therapeutics can be encapsulated in the nanoparticles to facilitate delivery of heme. Molecules such as hemoglobin and heme-HSA are carriers for the hydrophobic heme molecule. Both of these molecules appear in the blood and are inherently biocompatible. Heme-HSA was therefore selected as an investigational new therapeutic for AMD because dry AMD is triggered by inflammation and heme can overexpress HO-1 in other physiological systems, which reduces inflammatory markers. We have successfully loaded heme-HSA into the NPs and have initiated in vitro biocompatibility studies of heme-HSA with retinal cells. These nanoparticles can effectuate the sustained ROS-responsive release of heme-albumin to suppress inflammation in the retina. Thus, this platform has the potential to significantly improve treatment outcomes for patients with retinal degenerative diseases, particularly dAMD.

### Example 2: Release of Therapies from Responsive Nanoparticles to Treat Macular Degeneration

In this example, we further describe therapies and drug delivery systems for ocular diseases and injuries, including age-related macular degeneration (AMD).

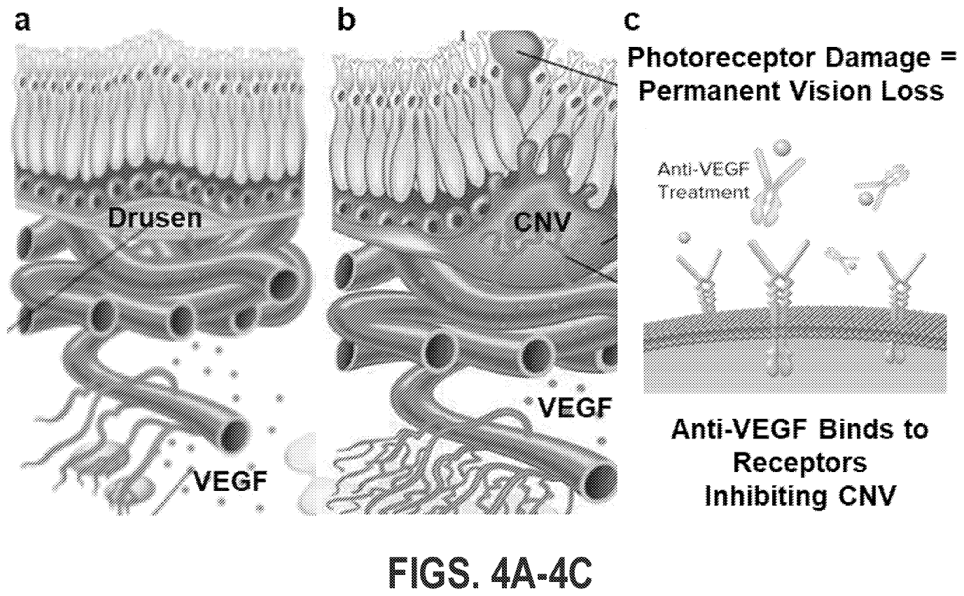

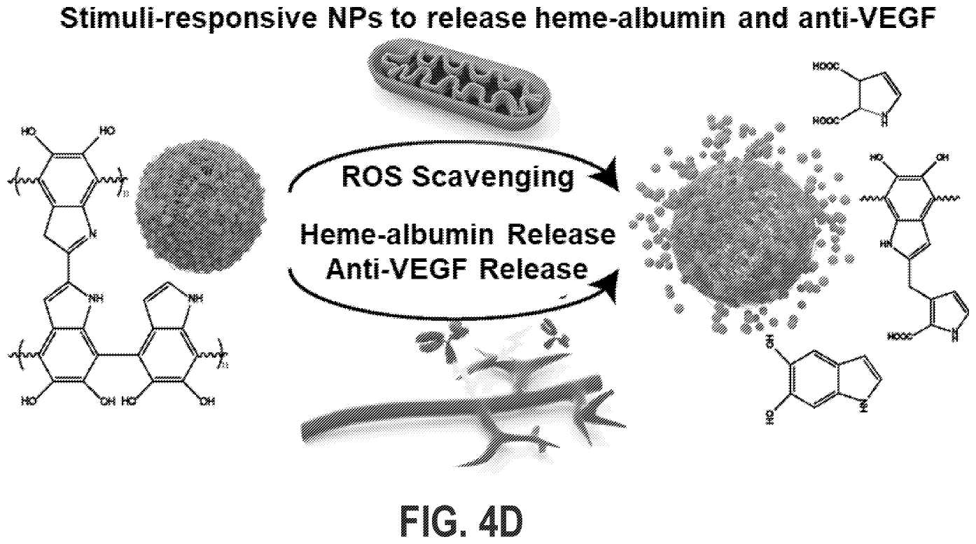

AMD is a retinal neurodegenerative disorder affecting central vision. It can lead to permanent blindness if left untreated, making it the leading cause of blindness in the US for individuals over 65 and the third leading cause of blindness worldwide. The local para-inflammatory response induced by reactive oxygen species (ROS) further contributes to excess expression of vascular endothelial growth factor (VEGF) which has been shown to play a significant role in the pathogenesis of AMD. ROS accelerate production of polyunsaturated fatty acids and cause protein aggregation, forming drusen in the dry (early) form of AMD. Wet (late-stage) AMD is characterized by choroidal neovascularization (CNV), the abnormal growth of leaky blood vessels, which further irreparably damages the retina. The introduction of anti-VEGF therapy has significantly improved outcomes for wet AMD by inhibiting new blood vessel formation, but requires frequent, often monthly, intravitreal (IVT) injections for maximum efficacy. There is currently no treatment for dry AMD (FIG. 4). Oral supplementation with antioxidants is the only known intervention that has been shown to reduce the progression of dry AMD by mitigating ROS. However, it requires daily pills with high doses of antioxidant vitamins to overcome the blood-retinal-barrier. Some of the high dose supplements can cause other health issues. Therefore, having a more local therapy to mitigate ROS would be desirable.

It should be emphasized that these current treatments cannot prevent or cure the disease. Instead, anti-VEGF therapies target angiogenesis without targeting the upstream inflammatory factors, which may limit their therapeutic effect. Moreover, these therapeutics are limited by short half-life and poor sustained release in the eye, requiring monthly intraocular injections that increase cost and burden on the healthcare system, lowers patient compliance, and potentially leads to poor clinical outcomes. Frequent IVT also comes with risk of adverse events including vision threatening endophthalmitis, inflammation, elevated intraocular pressure (IOP), retinal detachment, and cataract. Thus, there is a demonstrated clinical need for an improved therapeutic and delivery method to overcome the significant limitations in the current AMD treatment paradigm.

While IVT injection with anti-VEGF is invasive and typically yields a short retention time, it has several advantages in the eye, including decreased side effects on off-target sites, increased local drug concentration, and increased bioavailability. IVT is currently used clinically for long-term delivery of other therapeutics (e.g. Ozurdex, Iluvien). Recent publications have demonstrated potential for long-term delivery of anti-VEGF through intravitreally injected polymer systems for several months. These drug delivery approaches have largely focused on developing systems for delivery of anti-VEGF that can be injected intravitreally, and most have used poly (lactic-co-glycolic acid), which, while FDA approved, is known to have acidic degradation byproducts, which may lead to inflammation.

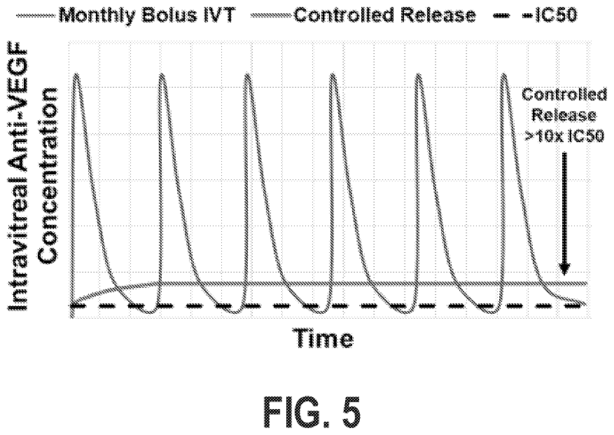

To address these limitations, we propose an injectable, biodegradable, redox-responsive controlled release system for AMD which will allow several months between injections and will treat two pathways of the disease. This has the potential to not only reduce issues associated with frequent injections, but can also reduce costs associated with administering frequent boluses of anti-VEGF well above the estimated half maximal inhibitory concentration (IC50) (FIG. 5). No IVT injected controlled release system is currently FDA approved for treatment of AMD.

Significantly, in addition to anti-VEGF, we will evaluate heme bound to human serum albumin (heme-albumin) as an investigational new therapeutic for AMD because dry AMD is triggered by inflammation and heme is known to overexpress heme-oxygenase-1 (HO-1) in other physiological systems, which then reduces inflammatory markers. Heme (616 g/mol) is hydrophobic and therefore insoluble in aqueous solution. Therefore, it distributes into lipophilic compartments (cell membranes and lipoproteins). The enzyme HO-1 converts heme into biliverdin, carbon monoxide (CO), and iron. In the cell, CO has potent anti-inflammatory properties Since AMD is associated with inflammation, it is proposed that delivery of heme to the eye should reduce inflammation via HO-1 catalyzed production of CO. Therefore, to enhance the aqueous solubility of heme for drug delivery applications, one can exploit the specific binding of heme to human serum albumin (HSA). Hence, in this work, the carrier protein HSA is used to store and transport heme to cells. HSA (66.5 kDa) has antioxidant properties, is responsible for approximately 80% of the colloid osmotic pressure (COP) of plasma, contains multiple hydrophobic molecule binding pockets, and naturally serves as a transporter of different ligands such as fatty acids, steroids, toxic species, drugs, and other molecules. HSA is synthesized by liver hepatocytes, and has a half-life of approximately 20 days In addition to its antioxidant properties, HSA inhibits inflammation during resuscitation, improves endothelial vascular integrity, and has no adverse effects on hemostasis. Therefore, HSA is a promising carrier protein for beme.

We incorporate therapeutics in a biodegradable, redox-responsive injectable nanoparticle (NP) system to extend release of therapeutic several months while adapting release rates in response to local ROS in the eye. Further, we will evaluate a new anti-inflammatory agent, heme-albumin, for treatment of both the dry and wet stages of the disease. The design of our NP delivery system has several significant advantages, including ability to sustain release, ability to trigger faster release in response to disease-associated stimuli, and anti-inflammatory byproducts of biodegradation. The poly (dopamine) (PDA) NPs biodegrade in the presence of ROS, which facilitates drug release by erosion in addition to diffusion. It is creative to study heme-albumin to reduce inflammation associated with dry and wet AMD. ROS-induced inflammation and angiogenesis have been considered critical pathogenic factors of AMD, making them two promising targets for therapy. We propose to suppress both factors concurrently through controlled therapeutic release and ROS scavenging. Incorporation of heme-albumin has potential to target inflammation that causes AMD, specifically by catalyzing production of CO through HO-1.

### Scientific Challenges

Biologic therapeutics, including anti-VEGF and heme-albumin, have a short half-life in the body and lose bioactivity over time in aqueous environments. This is particularly true in the vitreous humor (anti-VEGF half-life 6-9 days). Furthermore, these therapeutics are large (MW>48 kDa), complicating design of drug delivery systems. One scientific challenge is protecting bioactivity of therapeutics while sustaining local release. Another scientific challenge is ability to modulate release based on disease markers, particularly ROS An additional challenge is having a treatment for earlier stages of AMD, particularly for dry AMD. To address these challenges, we propose injectable stimuli-responsive NPs to enable localized and sustained delivery of bioactive anti-VEGF and anti-inflammator inside the eye. Further, the NPs use gentle chemistry which enables facile loading of therapeutics while preserving bioactivity and therapeutic efficacy. Our preliminary results demonstrate the ability to release large protein therapeutics including anti-VEGF at least 3-6 months in vitro while preserving bioactivity, with increased release rate in the presence of oxidative stress

### Clinical Challenges

Several diseases are caused by ROS, including heart disease, cataract, and diabetic retinopathy. AMD is the third leading cause of blindness worldwide, and the leading cause of blindness for individuals over age 65 in the US. The direct healthcare cost of AMD in the US is estimated at $5-10B. Global incidence of AMD is increasing and is continuing to rise, with the number affected in the US expected to double to 22 million by 2050. Quality of life is significantly decreased by visual impairment, primarily affecting reading and driving. With the number of patients requiring injections on the rise and recent evidence showing that lower anti-VEGF doses have efficacy when delivered in a controlled manner, this is pressing issue. It should be emphasized that there is no treatment for dry AMD and no cure for wet AMD-later stages must be managed with frequent injections over a patient's lifetime to prevent permanent vision loss. Despite control of wet AMD with anti-VEGF, vision continues to decline, potentially due to underlying dry AMD. The proposed treatment would address both arms of the disease. Clinical issues include therapeutic waste due to short half-life of drugs in the vitreous, and complications associated with frequent injections. Further, frequent injections are a burden to patients and caregivers, particularly for those who are unable to drive or live far from clinics. Developing an injectable, biodegradable drug delivery system with the ability to locally deliver anti-VEGF and anti-inflammatory therapeutics for several months will substantially lower barriers to more frequent treatment of a chronic disease, improving patient outcomes, and reducing cost to the healthcare system.

This approach has the potential to change the treatment paradigm for AMD, thereby improving patient outcomes while decreasing the burden on patients, clinicians, and the healthcare system. In this example, we will generate scientific knowledge on heme-albumin's ability to modulate inflammation in the eye, and whether there are synergistic effects with anti-VEGF. While the focus of this proposal is to validate the delivery system and heme-albumin as treatments for AMD, an injectable stimuli-responsive system capable of protecting and delivering therapeutics long-term has potential applications in other ocular and inflammatory diseases. In addition to anti-VEGF and heme-albumin, this system has the potential to be used for controlled delivery of other therapeutics in combination with its ROS scavenging effects. Taken together, the therapeutic approach in this project has the potential to pave a new road for combinatorial therapy to improve patient outcomes while simultaneously decreasing injection frequency.

### Innovation

We have developed a stimuli-responsive NP delivery system to specifically release more therapeutic in response to oxidative stress. The preparation technique allows for gentle loading chemistry that preserves therapeutic bioactivity, overcoming one significant barrier for sustained release of proteins and antibodies.

We have developed an injectable drug delivery system that is unlikely to induce inflammation, as PDA has anti-inflammatory byproducts of degradation. These characteristics help the system go beyond simply sustaining release, overcoming significant limitations with the current treatment paradigm and other systems.

We have prepared heme-albumin and propose to evaluate this novel therapeutic for treatment of inflammation associated with AMD. The heme-albumin complex will deliver heme to cells in the eye. Cellular HO-1 will convert heme into biliverdin, iron, and CO. The CO generated is a highly anti-inflammatory molecule, and has the potential to resolve the inflammation present in AMD. Therefore, this approach presents a natural mechanism for the generation of a potent anti-inflammatory molecule for treatment of inflammation in AMD.

The proposed system has potential for synergistic treatment of inflammation and angiogenesis in AMD. Inhibition of neovascularization through both VEGF and VEGF-independent pathways is additionally proposed for treatment.

### Approach

The rationale for this study is that controlled release of anti-inflammatory and anti-angiogenic therapeutics coupled with a biodegradable stimuli-responsive drug delivery system will significantly improve outcomes for patients currently requiring frequent injections to preserve vision and manage AMD. We hypothesize that heme-albumin will induce retinal cells to overexpress HO-1, reducing inflammatory markers. We further hypothesize the NP delivery system will release and sustain bioactivity of heme-albumin alone or with other therapeutics such as anti-VEGF at least three months and will release the therapeutics at a faster rate when exposed to higher levels of ROS. This has the potential to facilitate treatment of two mechanisms of AMD. We will evaluate the capability of the system to 1) scavenge disease-inducing ROS, 2) controllably release anti-angiogenesis and novel anti-inflammatory therapeutics, and 3) extend the therapeutic life-span and release.

### Preliminary Data

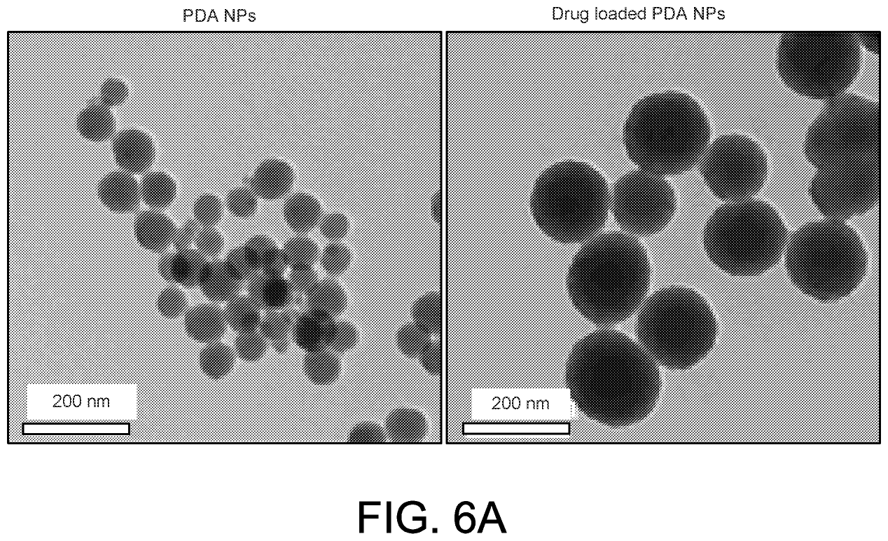

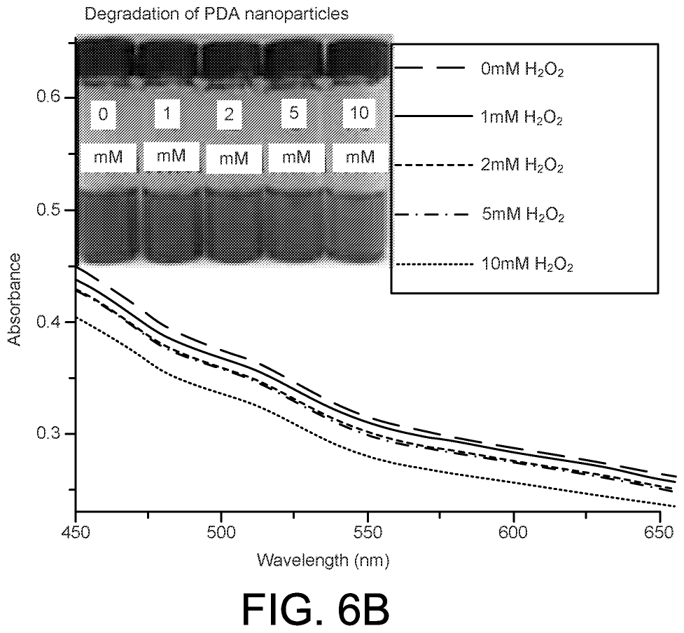

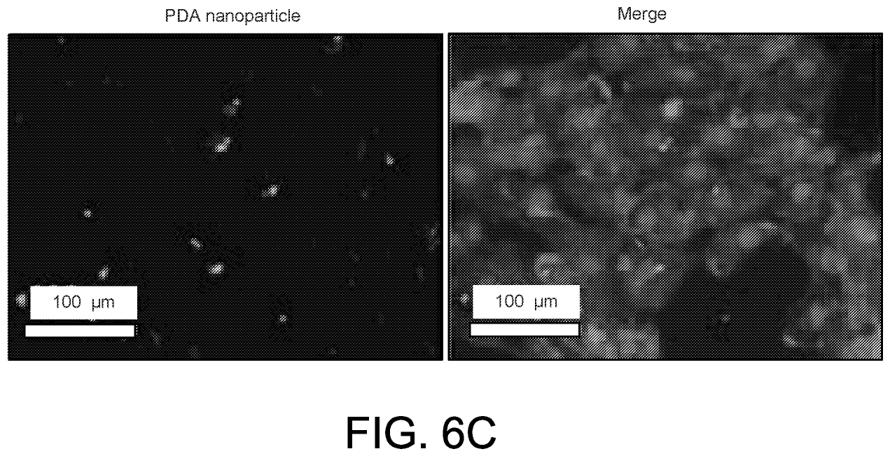

We have developed anti-VEGF loaded PDA NPs (FIGS. 4A-4D and 6A-6C) with the potential to treat AMD by inhibiting both ROS and VEGF. The building block of the NPs, dopamine, is naturally prevalent in the human body. The rationale for using PDA as an antioxidant is that it can inhibit both acute and para inflammation by capturing heavy metal ions and scavenging ROS via its abundant phenol groups. Moreover, the cavities on the NP surfaces can efficiently load a high concentration of drugs including larger protein therapeutics such as anti-VEGF and heme-albumin (heme-HSA), and have potential for long-term therapeutic delivery.

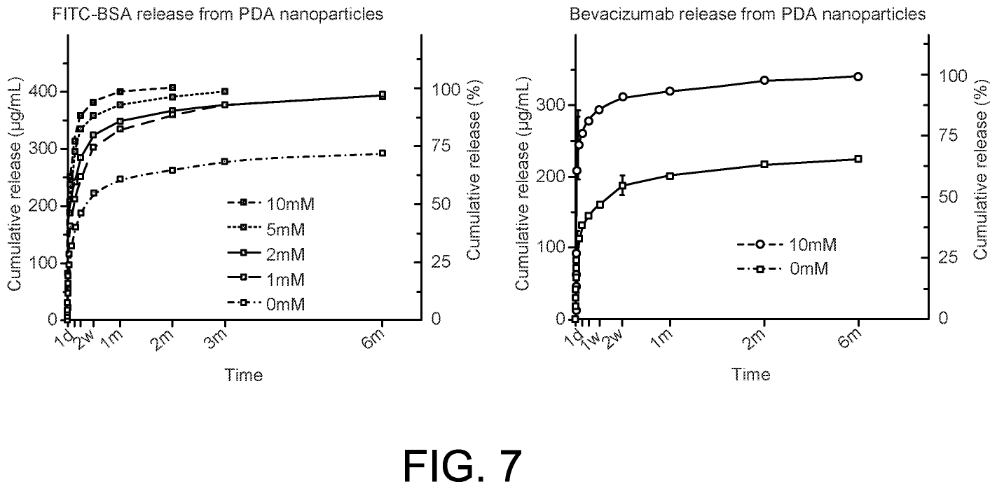

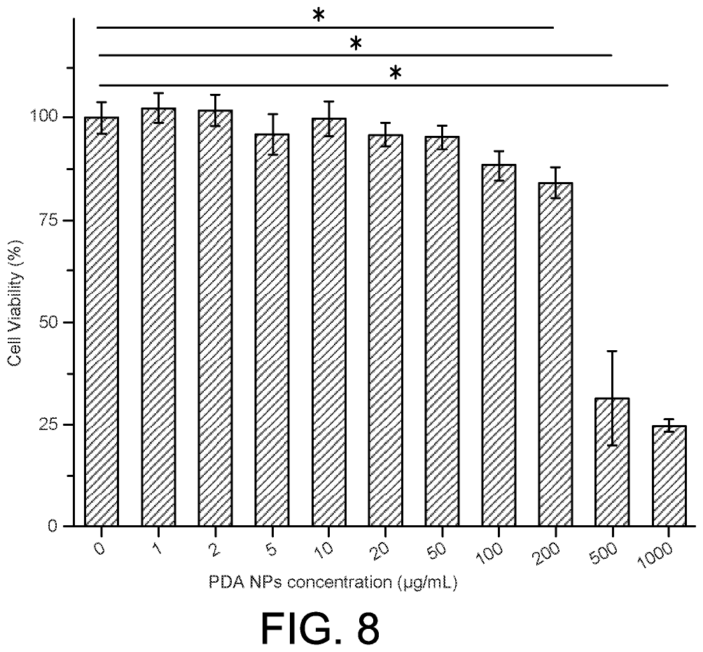

PDA NPs have been synthesized and loaded with anti-VEGF (149 kDa) and model therapeutic bovine serum albumin (BSA) (66.5 kDa), which has similar properties to heme-HSA (67 kDa). These protein-loaded NPs are 160 nm in diameter, an optimal size for intraocular injection through a small needle (FIG. 6A). These NPs were easily injected into ex vivo porcine eyes obtained from a local abattoir through a 31 G needle. The reaction of PDA NPs with H₂O₂ to stimulate ROS increased degradation (FIG. 6B), and NPs loaded with FITC-BSA (green) were imaged inside cells labeled with DAPI (blue) and phalloidin (red) (FIG. 6C). In preliminary studies, in vitro release of model protein BSA and anti-VEGF (bevacizumab) were evaluated. Drug loading capacity was of 35-40% of the NPs. NPs sustained release of BSA 2-6 months and anti-VEGF at least 3 months in vitro, as determined by BCA and enzyme-linked immunosorbent assays (ELISA). Therapeutics were not completely released from NPs at low levels of oxidative stress at the end of the study (FIG. 7) In vitro cytotoxicity was determined by MTS assay (FIG. 8) and live/dead assay with human retinal pigment epithelial cells (ARPE-19). No significant cell death was observed at NP concentrations from 0 to 50 μg/mL, and NPs entered the cells.

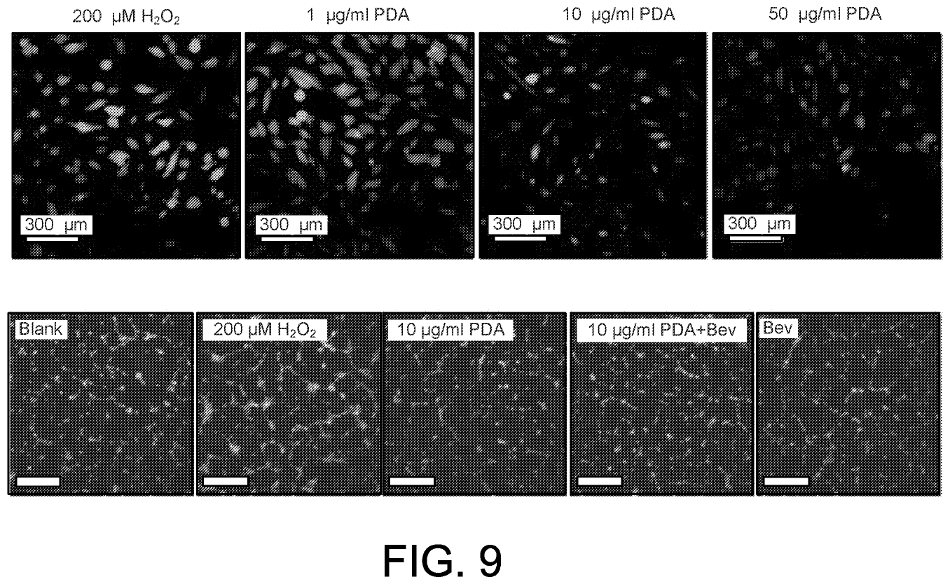

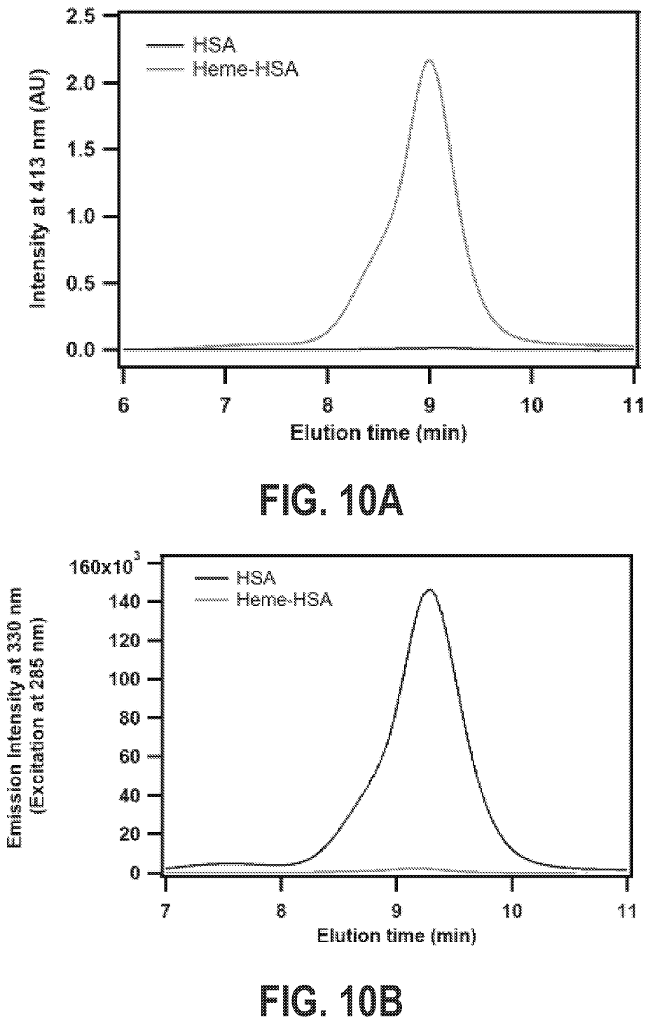

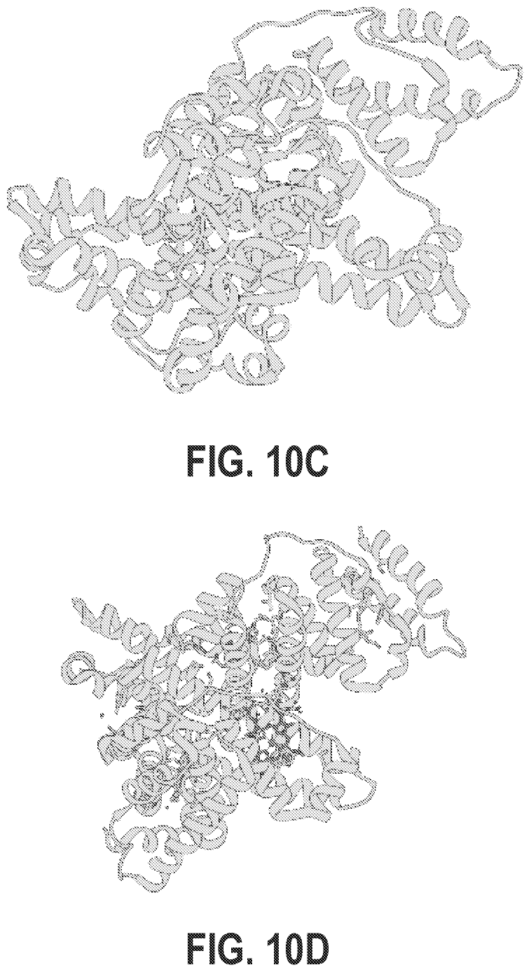

Under oxidative stress induced by 200 μM H₂O₂, ARPE-19 cells were co-cultured with human umbilical vein endothelial cells (HUVECs) to investigate the influence of H₂O₂ and drug-loaded PDA NPs on the development of the three-dimensional capillary structure. Exposure to H₂O₂ induced secretion of VEGF and tubule formation. Cells were then incubated with 5 μg/mL native bevacizumab control or 10 μg/mL NPs with and without anti-VEGF. As shown by Calcein AM staining in FIG. 9, both the blank NPs and anti-VEGF loaded NPs significantly inhibited tubule formation in HUVECs compared to the positive control. The reaction of PDA NPs with H₂O₂ also simultaneously degraded (FIG. 6B) and triggered drug release (FIG. 7), favorable for spatiotemporal control of release at disease sites.

While preliminary data demonstrates release of anti-VEGF, AMD is also characterized by inflammation, which is not resolved by anti-angiogenic therapeutics. Therefore, delivery of anti-inflammatory drugs to the eye is a reasonable strategy to resolve AMD-associated inflammation. Fortunately, heme can be metabolized in cells by the enzyme HO-1 to yield anti-inflammatory byproducts. Since heme is not soluble in aqueous media, we have developed several heme-containing protein therapeutics. Molecules such as hemoglobin and heme-albumin are carriers for hydrophobic heme. Both of these molecules appear in the blood and are inherently biocompatible. Heme-HSA was therefore selected as an investigational therapeutic for AMD because dry AMD is triggered by inflammation and heme overexpresses HO-1 in other physiological systems, reducing inflammatory markers. FIGS. 8A-8D below show our ability to successfully incorporate heme into HSA. These preliminary studies demonstrate the ability to prepare both heme-HSA and stimuli-responsive PDA NPs to modulate drug release.

### Example 3: Sustained Release of Heme-Albumin as a Potential Therapeutic Approach for Age-Related Macular Degeneration

### Overview

Globally, age-related macular degeneration (AMD) is the third most common visual impairment. Most often attributed to cellular fatigue with aging, over expression of reactive oxygen species (ROS) accumulates in the retina, leading to chronic inflammatory immune signaling, cellular and tissue damage, and eventual blindness. If left uncontrolled, the disease will progress from the dry form of AMD to more severe forms such as geographic atrophy or wet AMD, hallmarked by choroidal neovascularization. There is no cure for AMD and treatment options are limited. Treatment options for wet AMD require invasive ocular injections or implants, yet fail to address the disease progressing factors. To provide more complete treatment of AMD, the application of a novel anti-inflammatory heme-bound human serum albumin (heme-albumin) protein complex delivered by antioxidant ROS scavenging polydopamine (PDA) nanoparticles (NPs) for sustained treatment of AMD was investigated. Through the induction of heme oxygenase-1 (HO-1) by heme-albumin in retinal pigment epithelial (RPE) cells, anti-inflammatory protection may be provided through the generation of carbon monoxide (CO) and biliverdin during heme catabolism Our results show that the novel protein complex has negligible cytotoxicity towards RPE cells (ARPE-19), reduces oxidative stress in both inflammatory and ROS in vitro models, and induces a statistically significant increase in HO-1 protein expression. When incorporated into PDA NPs, heme-albumin was sustainably released for up to 6 months, showing faster release at higher oxidative stress levels. Through its ability to react with ROS, heme-albumin loaded PDA NPs showed further reduction of oxidative stress with minimal cytotoxicity. Altogether, we demonstrate that heme-albumin loaded PDA NPS reduces oxidative stress in vitro and can provide sustained therapeutic delivery for AMD treatment.

### Introduction

Age-related ocular disorders continue to increase in prevalence as the average global lifespan continues to grow, with the third leading cause of blindness relating to age-related macular degeneration (AMD).¹ Today, the prevalence of AMD, globally, affects more than 196 million people with the projected number of people afflicted to increase to 288 million by 2040, with the United States alone accounting for over 11 million cases in patients over 60.²³

Disease progression of AMD comes in two forms: early and late stage. Early or dry AMD is the most common form of the disease, accounting for almost 90% of cases and is hallmarked by intracellular or extracellular protein aggregation and toxic debris, resulting in lipofuscin and drusen formation, respectively, lipid oxidation, and chronic inflammation in the region.⁴⁻⁶ Advanced dry AMD or geographic atrophy (GA) results in degradation of the macular and retinal tissues, spreading to cover significant fractions of posterior segment tissues, and leading to partial or complete vision loss.⁷ There are currently no approved treatment strategies for dry AMD. Alternatively, late stage or wet AMD is the more advanced version of the disorder, found in 10% of cases and is characterized by increased production of vascular endothelial growth factor (VEGF) leading to new vascularization in the choroid.⁴ The sub-retinal blood vessels breach the integrity of the Bruch's membrane into the retinal pigment epithelium (RPE), causing permanent damage to photoreceptor cells, RPE tearing and detachment, sub-retinal hemorrhage, fibrotic scarring, and permanent vision loss.^{8,9}

Factors attributed to disease progression in both types of AMD include: age, location and sunlight exposure, diet, smoking status, and demographics.¹⁰ Common among these contributors is their impact on generation of oxidative stress within the posterior segment of the eye and the damaging effects to ocular tissues.¹¹ Metabolic imbalances between prooxidative and antioxidant regulation allow excess production of reactive oxygen species (ROS) which accumulate in the retina.¹²⁻¹⁴ ROS react with the surrounding proteins, lipids, and cellular DNA, causing damage and further generation of ROS, beginning a deteriorating cycle for the involved tissue.^{12,14} While the exact understanding of early AMD propagation is not confirmed, research has shown clear correlation supporting the relationship between oxidative injury to the RPE and surrounding tissues and cells, and the association of lipoproteins and parainflammatory immune responses within the retina.^{15,16}

There is currently no clinically available cure for either dry or wet AMD. Early dry AMD treatments consist mostly of regular observation and documentation for early signs of neovascularization. More commonly, treatment of wet AMD includes frequent, up to monthly, intravitreal injections of anti-VEGF therapeutics to inhibit further growth factor binding and neovascularization.¹⁷ The invasive and costly injections are required for the rest of patient's lives and often experience low patient compliance and under treatment of the disease.¹⁸ Additionally, the injections increase the patient's risk for injection related complications such as endophthalmitis, thegmatogenous retinal detachment, and increases in ocular inflammation and pressure.¹⁹ Intraocular implants, specifically Genentech's SUSVIMO®, is a newly approved extended port delivery system that is capable of providing 6 months of continued anti-VEGF application with minimally invasive refilling of the drug reservoir Despite the improved injection frequency, the device requires surgical implantation and has shown a high incidence rate of endophthalmitis, generally 3 times higher than the rate for traditional intravitreal injection of anti-VEGF.²⁰ Intravitreal injection of anti-VEGF treatments for patients with dry AMD are uncommon as they have not shown the same success at mitigation of disease progression as when used for wet AMD.⁶ Furthermore, links between extensive anti-VEGF use and exacerbation of retinal and macular atrophy have been seen in patients with dry AMD.^{21,22} Newly emerging investigative treatments for dry and wet AMD include cell-based therapies for regrowth of retinal cells, viral vector gene therapy, and complement cascade inhibition to mitigate inflammatory signaling and toxic complement by-products that are largely associated with disease progression.^{23,24}

Utilization of the natural oxidative stress reduction pathways for treating inflammation and ROS remains an unexplored therapeutic approach for treating AMD. Within this response, heme oxygenase-1 (HO-1) has been found to be an essential protein in combating oxidative stress and inflammation.²⁵ HO-1 maintains intracellular heme levels through the catabolismof the pro-oxidative protoporphyrin into the anti-inflammatory and antioxidant molecules biliverdin and carbon monoxide (CO).²⁶ The generation of both CO and biliverdin provide essential mediation of the oxidative stressing signals produced by immune cells. Pro-inflammatory cytokines activate expression of p38 mitogen associated protein kinase (p38 MAPK), a transcription factor important in induction of HO-1, with and β isoforms, that present pro-apoptotic or anti-apoptotic impacts, respectively.^{27,28} HO-1, and the production of CO, increases expression of the p38β isoform resulting in upregulation of anti-inflammatory IL-10 expression with simultaneous downregulation of pro-inflammatory cytokines.^{26,27} Additionally, conversion of biliverdin to bilirubin is catalysed via the biliverdin reductase (BVR) enzyme pathway. Bilirubin has shown promising antioxidant effects through ROS reduction, leading to its conversion back to biliverdin, providing a continual antioxidant protective loop.²⁹⁻³¹

We therefore hypothesize that intentional and specific induction of HO-1 for therapeutic relief shows potential as a treatment for AMD. HO-1 has shown to be expressed in RPE cells and would be responsive during oxidative assault in AMD.^{32,33} To induce expression of the immune enzyme and generate the anti-inflammatory and antioxidant by-products, we propose the application of heme bound to human serum albumin (heme-albumin) to activate expression of HO-1. Additionally, HSA has shown antioxidant properties of its own as a major source of reactive free thiols that can scavenge ROS and its high affinity for dangerous free transition ions.³⁴ Thus as a synthesized protein complex, heme-albumin would be a dual threat therapeutic to combat the oxidative stress of AMD that leads to disease application.

Direct application of heme-albumin, through intravitreal injection, would be inadequate to provide sustained, continued treatment against AMD due to rapid drug clearance in the vitreous.³⁵ Therefore, polydopamine (PDA) nanoparticles (NPs) will be utilized for delivery of heme-albumin as an easily synthesized, and mucoadhesive drug delivery system for treatment of AMD. Recent studies in imaging, periodontal, and autoimmune disorder treatment applications, show that the self-polymerizing NPs are capable of ROS scavenging, providing additional antioxidant benefits in combating oxidative stress in AMD, without inducing cytotoxicity.³⁶⁻³⁸

We therefore hypothesize that heme-albumin, a protein therapeutic utilizing the naturally occurring beme molecule, delivered by ROS scavenging PDA NPs, will thoroughly and effectively combat both inflammation and ROS induced oxidative stress in retinal pigment epithelial cells, potentially providing a more complete defense against the perpetuating factors of both forms of AMD.

### Materials and Methods

Materials. Hemin, dopamine hydrochloride, heme assay kit, fortified bovine calf serum, bovine serum albumin (BSA), 10% neutral buffered formalin, and Dulbecco's phosphate buffered saline (DPBS), was purchased from Sigma-Aldrich (St. Louis, MO). Human serum albumin (HSA), 25 mg/mL was obtained from Octapharma® (Lachen, Switzerland). Hollow fiber tangential flow filtration (TFF) modules (D01-S050-05-N, polysulfone membrane, 50 kDa pore size, 60 individual hollow fibers, 0.5 mm internal diameter, 190 cm² total surface area) were purchased from Repligen (Rancho Dominguez, CA). TFF system tubing and peristaltic pump (Masterflex L/S® precision pump tubing, EW-96410-16 and Masterflex L/S® Digital Drive with Easy-Load® 3 pump head, EW-77921-65) were obtained from Cole-Palmer (Vernon Hills, IL). Tube connections were acquired from Nordson Medical (Loveland, CO). Phosphoric acid (H₃PO₄), sodium chloride (NaCl), sodium hydroxide (NaOH), sodium phosphate monobasic (NaH₂PO₄) and dibasic (Na₂HPO₄), Triton X-100, and 0.2 μm Titan3 sterile filters were obtained from Fisher Scientific Inc. (Hampton, NH), and Dulbecco's Modified Eagle Medium (DMEM) F-12was purchased from Thermo Fisher Scientific (Waltham, MA). HyClone Penicillin-Streptomycin³⁹⁻⁴¹ 100× solution and trypsin 0.05% (1×) were obtained from Cytiva (Marlborough, MA). Human retinal pigment epithelial cells (ARPE-19 cells, CRL2302) were purchased from American Type Culture Collection (ATCC, Rockville, MD). Colorimetric (3-(4,5-dimethylthiazol-2-yl)-5-(3-carboxymethoxyphenyl)-2-(4-sulfophenyl)-2H-tetrazolium) (MTS) assay, DCFDA/H2DCFDA Cellular ROS Assay Kit, and human heme oxygenase 1 ELISA kit were procured from Abcam (Cambridge, United Kingdom). Pierce™ BCA Protein Assay Kit, p38β MAPK antibody (p38-1145), IL-1β and phospho-p38 MAPK ELISA kit, 4′,6-diamidino-2-phenylindole (DAPI), α-mouse Alexa Fluor 568 secondary antibody, and human PCR primers (IL-6, IL-1β, and GAPDH,) along with SuperScript™ VILO™ cDNA Synthesis Kit were purchased from Thermo Fisher Scientific Inc (Waltham, MA). Additional primers for IL-1β, IL-6, caspase-3, and caspase-9 were purchased from Integrated DNA Technologies (Coralville, IA)

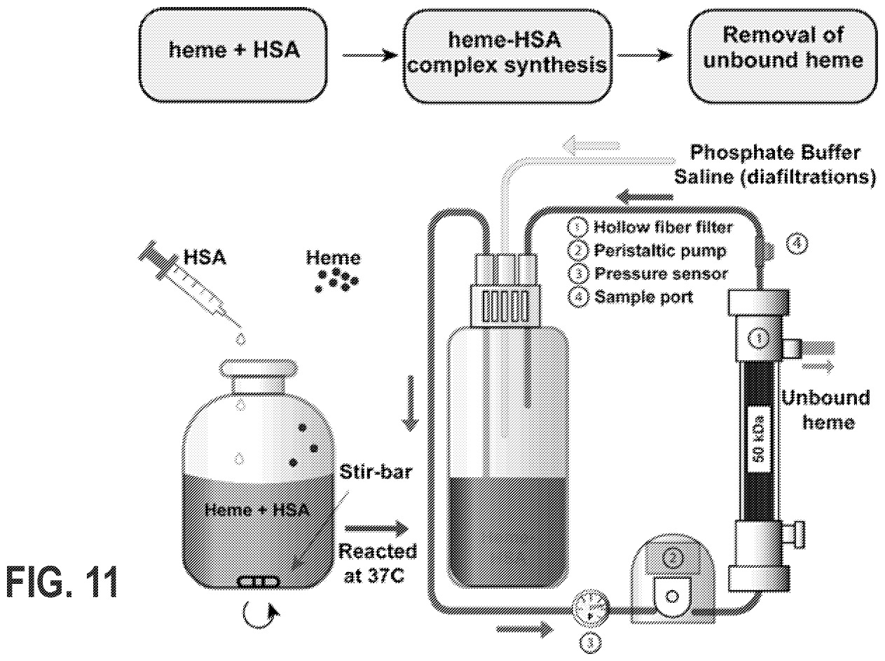

Heme-Albumin Synthesis and Purification. A 3 mM solution of heme was solubilized in 100 mM NaOH and combined with a 1.5 mM solution of HSA. Heme stability in various non-aqueous conditions, including NaOH, has been verified in previous studies.³⁹⁻⁴¹ The solution was mixed briefly before incubation at 37° C. for 1 hour after which the pH was adjusted to 7.4 with a phosphoric acid/NaCl solution and sterile filtered through a 0.2 μm filter. To remove any unbound heme, the heme-albumin protein complex was buffer exchanged over a 50 kDa TFF hollow fibre filter into phosphate buffered saline (PBS, pH 7.4). A schematic of the heme-albumin synthesis and buffer exchange process is shown in FIG. 11. Heme bound to albumin was quantified with a heme assay kit (n=5), where 50 μL of a blank, 50 μL of heme calibrator, and 50 μL of diluted heme-albumin were added to individual wells in a 96-well clear plate. To each well, 200 μL of heme reagent was added. The contents of each well were mixed and left to react for 5 minutes at room temperature before the optical density was measured at 400 nm. Protein quantification was performed using a Dionex UltiMate 3000 UHPLC/HPLC system with an Acclaim SEC-1000 (4.6×300 mm) column with a 50 mM phosphate buffer mobile phase pH 7.4. Chromeleon 7 software was used to control and measure HPLC parameters such as flow rate (0.35 mL/min), UV-visible absorbance detection (280 nm) and fluorescent light detection (excitation/emission (Ex/Em) at 285/333 nm). All samples were filtered through 0.2 μm syringe filters before analysis. The absorbance at 280 nm was used for total protein quantification of the heme-albumin complex, whereas confirmation of heme integration into albumin was determined by fluorescence quenching of heme at 285/333 nm compared to native albumin.

Circular Dichroism (CD) of Heme-Albumin. The secondary structure of heme-albumin was investigated with a Jasco J-815 CD spectrometer (Easton, MD). The spectra was taken from 180-250 nm, 0.5 nm step, with a 0.1 cm quartz cuvette. Baseline correction was employed and sample concentrations of 0.1-0.2 mg/mL were used for analysis.

Heme-Albumin Loaded Polydopamine Nanoparticle Synthesis. Dopamine hydrochloride precursor was reacted with a polymerization initiator solution composed of 45 mL deionized water (DI H₂O) with 380 μL I N NaOH as published.⁴² The precursor was added to a 250 mL round bottom flask and initiator was added at a rate of 4 mL/min after which the self-polymerization reaction was left to proceed for 3 hours with constant stirring. The solution pH was adjusted to pH 7.4 before the addition of 100 mg of 2:1 heme-albumin protein complex. After addition, protein loading was allowed to continue for 21 hours before centrifugation at 12,100 rpm for 15 minutes and washing 3× times with DI H₂O. The heme-albumin loaded PDA NPs were lyophilized at −88° C. and 0 002 mbar for 24 hours. Unloaded PDA NPs were centrifuged, washed, and lyophilized using the same procedure and were collected before protein addition.

Nanoparticle Characterization. Lyophilized nanoparticles were resuspended in DI H₂O at 1 mg/mL and characterized by a FEI Tecnai G2 Spirit transmission electron microscope (TEM) (Thermo Fisher, Waltham, MA) with 1% uranyl acetate. Scanning electron microscopy (SEM) of both the loaded and unloaded PDA NPs were conducted on a Thermo Fisher Scientific Apreo LoVac UXR (Waltham, MA). Lyophilized NP samples were fixed on carbon graphite tape before image capture. The hydrodynamic diameter and zeta potential of the nanoparticles was measured using a BI-200SM Goniometer and ZetaPals Zeta Potential Analyzer (Brookhaven Instruments Corp., Holtsville, NY). Dynamic light scattering (DLS) was performed at an angle of 90° and a wavelength of 637 nm. The hydrodynamic diameter was obtained by using the average values from the nonlinear least-squared algorithm in the instrument software. A total of n=10 measurements were taken by ZetaPals and the value was averaged to determine the zeta potential of the nanoparticles.

In Vitro Protein Release. A 4 mL solution of heme-albumin loaded polydopamine nanoparticles at a nanoparticle concentration in solution of 1 mg/mL was incubated at 37° C. at varying concentrations of oxidative stressor: 0, 0.5, and 1 mM H₂O₂ in DPBS for investigation of sustained release of heme-albumin. At time points 1 day, 4 days, 1 week, 2 weeks, 1 month, and monthly for up to 6 months, nanoparticles were collected by centrifugation at 20,000 rpm for 20 minutes before retrieval of all the supernatant and 4 mL of replacement oxidant solution was added to maintain sink conditions. Supernatants were stored at 4° C. and analysed by BCA protein quantification assay.

Cell Culture. ARPE-19 cells were cultured with DMEM/F-12 media supplemented with 10% fetal bovine serum and 1% penicillin-streptomycin 100× solution. Media was exchanged every 1-3 days and cells were passaged at 80-90% confluence. At passaging, cells were washed with 10 mL DPBS before addition of 3-5 mL of 0.05% trypsin and incubated at 37° C. for 5 minutes. Cells were visualized under a microscope to confirm non-adherence and trypsin was neutralized with the addition of 5-7 mL of media. Cells were pelleted at 130 g for 7 minutes before media was aspirated and resuspended for plating.

Cell Viability Assay. The cytotoxicity of heme-albumin, PDA NPs, and heme-albumin PDA NPs were investigated using ARPE-19 cells. The cells were seeded at 4,000 cells/well in a clear 96-well plate.⁴² After 24 hours, to confirm cell adherence, varying concentrations of heme-albumin (10-2000 μg/mL) or NP concentrations, both unloaded and loaded (10-200 μg/mL) were applied to the wells and left to incubate for an additional 24 hours. Media contacting the therapeutic was removed and MTS stain was applied. The cells were stained for 3 hours before measurement of optical density at 490 nm with Varioskan™ Lux multimode microplate reader using SkanIT software (Thermo Fisher Scientific, Waltham, MA).

Oxidative Stress Assay. The ability of heme-albumin, PDA NPs and heme-albumin loaded PDA NPs to reduce oxidative stress in both inflammatory and ROS models in ARPE-19 cells was investigated using published techniques with slight modifications.⁴² Cells were seeded at a density of 4×10⁴ cells per well in a 96-well dark walled plate and left to adhere for 24 hours. Therapeutic agent heme-albumin, blank PDA NPs, or heme-albumin loaded PDA NPs were applied at varying concentrations (10-1000 μg/mL or 10-200 μg/mL for both nanoparticle groups, respectively) to the cells directly. In addition to the therapeutic, lipopolysaccharide (LPS) (10 μg/mL) or H₂O₂ (100 μM) were applied to the wells. After 24 hours of incubation with therapeutic and stressing agent, the cells were washed with 100 μL 1× dilution buffer, stained for 45 minutes with 100 μL of 10 μM DCFDA dye and washed again with dilution buffer before fluorescence at Ex/Em=485/535 nm was measured with the microplate reader.

ELISA Quantification. The ability of here-albumin to induce expression of HO-1, under baseline and stressed conditions, was quantified using a heme oxygenase 1 ELISA kit. Heme-albumin's impact on IL-1β and activated p38 MAPK in retinal cells were also measured. Briefly, ARPE-19 cells were seeded in 6-well plates at 1×10⁶ cells/well (n=6). Cells were allowed to adhere 24 hours before aspiration of media and treatment with either 500 μg/mL heme-albumin, 100 μM H₂O₂ or co-treated with heme-albumin and 100 μM H₂O₂. Fresh media was used as a basal control. Incubation with treatment was continued for 24 hours before analysis. Cell lysis and ELISA were performed via manufacturer's recommendation.

PCR Quantification. Investigation of heme-albumin's effect on baseline expression of pro-inflammatory cytokines, compared to control, or heme-albumin's impact on gene expression in oxidative challenge conditions was analysed in an identical manner as described above. 1×10⁶ ARPE-19 cells/well were seeded in a 6-well plate (n=6) and treated with 500 μg/mL of heme-albumin after 24 hours. Media was used as a baseline control. The samples were collected and isolated with Tryzol. After RNA quantification, the cDNA was made using SuperScript™ VILO™ cDNA Synthesis Kit. The total mass used to prepare the cDNA was 2500 ng. 50 ng of cDNA was used to perform the PCRs. Human IL-6, IL-1β, Caspase-3, and Caspase-9 were used as target genes for investigation of heme-albumin's impact on pro-apoptotic and inflammatory cytokine expression. Primer sequences for PCR are supplied in Table 1 below. GAPDH was used as a housekeeping gene.

TABLE 1
Gene expression primers used for IL-6, IL-1β, Caspase-9, and Caspase-3.

| Primer name | Forward sequence 5′-3′ | Reverse sequence 5′-3′ |
|---|---|---|
| Human-IL6 | TCGGTCCAGTTGCCTTCTC | GAGGTGAGTGGCTGTCTGT |
| | (SEQ. ID: 1) | (SEQ. ID: 2) |
| Human-IL1B | CTCGCCAGTGAAATGATGGCT | GTCGGAGATTCGTAGCTGGAT |
| | (SEQ. ID: 3) | (SEQ. ID: 4) |
| Human-Caspase 9 | GTTTGAGGACCTTCGACCAGCT | CAACGTACCAGGAGCCACTCTT |
| | (SEQ. ID: 5) | (SEQ. ID: 6) |
| Human-Caspase 3 | GGAAGCGAATCAATGGACTCTGG | GCATCGACATCTGTACCAGACC |
| | (SEQ. ID: 7) | (SEQ. ID: 8) |

p38β MAPK Immunofluorescent Staining. Changes in production of the p38β MAPK protein were measured by immunofluorescent staining. ARPE-19 cells were seeded in a 12-well plate before treatment with either 100 μM H₂O₂ or co-treatment with 100 μM H₂O₂ and 500 μg/mL of heme-albumin (n=4) Cells without either treatment were used as a negative control. After 24 hour incubation with treatments, cells were fixed with 0.1% Triton X-100 for 10 minutes, blocked with 5% BSA for 1 hr, and stained with p38 β MAPK primary antibody, in 1% BSA, overnight. Mouse IgG secondary antibody was added before DAPI staining.

Statistical Analysis. All statistical analysis was performed using one-way ANOVA with Tukey honestly significant difference (HSD) test in JMP software. Results were considered statistically significant for p≤0.05.

### Results and Discussion

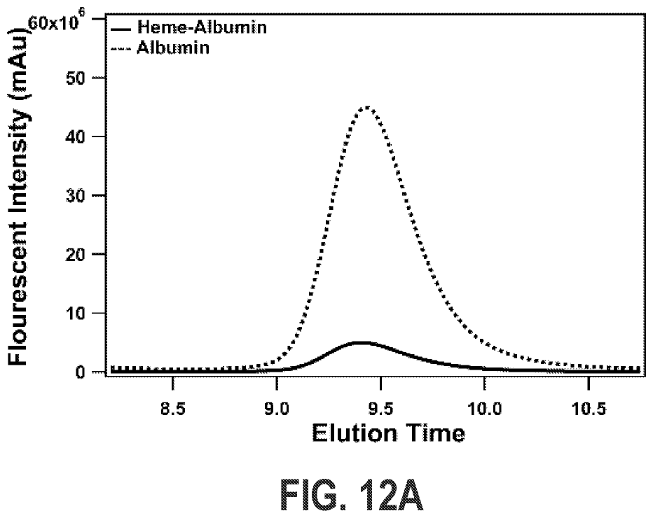

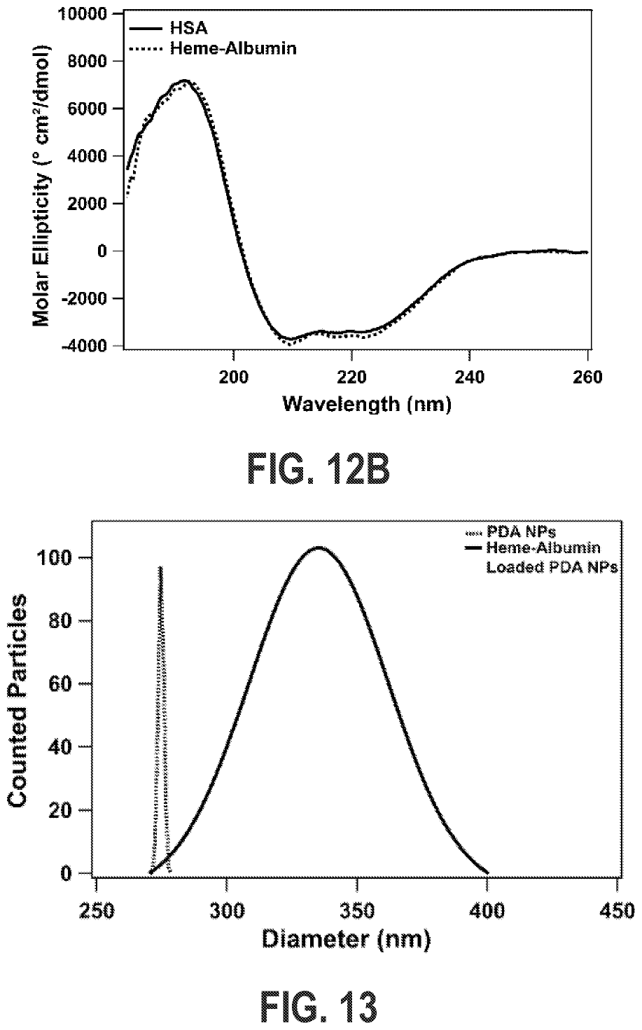

Heme-Albumin Characterization. Heme and albumin concentrations were quantified separately after TFF purification of heme-albumin by heme assay kit and SEC-HPLC, respectively, to determine the working molar ratio of 2.1 heme:HSA. Heme binding to albumin was determined by SEC-HPLC and is presented in FIGS. 12A-12B. Incorporation of beme into albumin quenches the native fluorescence of albumin. Measured at Ex/Em=285/330 nm, unbound HSA has a natural fluorescence, due to the aromatic amino acids, tryptophan and tyrosine.⁴³ When heme binds to the protein, the fluorescence is substantially quenched due to the interaction of the heme molecule and fluorescence contributing amino acids, verifying the formation of the heme-albumin protein complex. Heme binding to albumin resulted in a slight increase in molecular weight of heme-albumin compared to HSA, resulting in a small change in the protein elution time as seen in FIG. 12A with a small left shift of the heme-albumin peak compared to HSA. The secondary structure of heme-albumin compared to HSA was examined with CD and showed no significant deviation from the native HSA secondary structure. CD spectra was measured from 180-260 nm and presented as molar ellipticity shown in FIG. 12B. Unfolding under basic conditions allows for heme incorporation into the hydrophobic binding sites within the protein that are entrapped within after pH adjustment back to physiological pH.

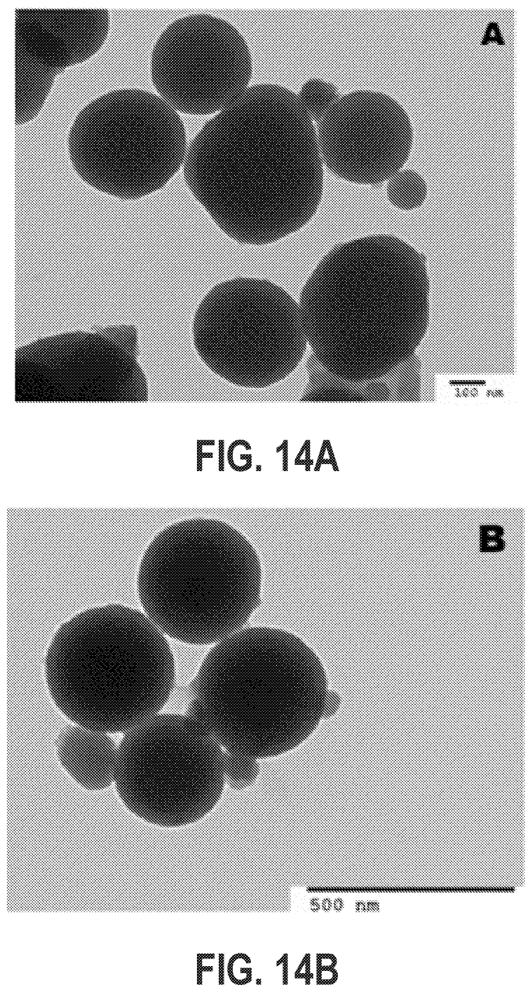

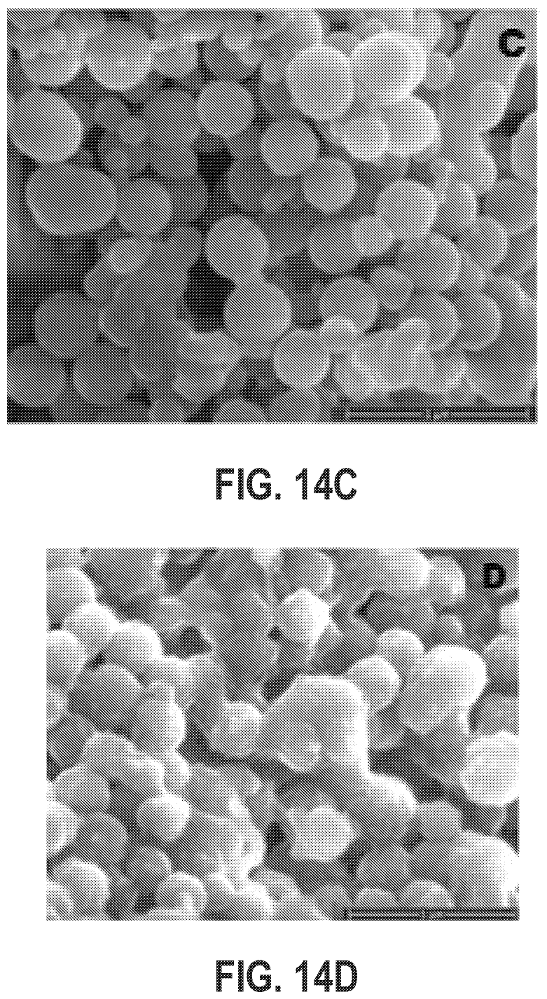

Polydopamine Nanoparticle Characterization. Both unloaded and heme-albumin loaded PDA NPs were characterized to determine the morphology and properties of the drug delivery devices. Unloaded PDA NPs possessed a spherical morphology that remained when loaded with heme-albumin. Particles without protein had a mean hydrodynamic diameter of 189 nm and measured 296.4±40.6 nm through TEM sizing. Both diameters are larger than previously reported nanoparticles with similar synthesis procedures but within the range of size suitable for use in drug delivery.⁴² Heme-albumin loaded PDA NPs possessed a hydrodynamic diameter of 229 nm and a measured diameter by TEM analysis of 307.5±27.9 nm. TEM and SEM images of unloaded and heme-albumin loaded PDA NPs are shown in FIG. 14A-14D and allow for confirmation of the spherical morphology of the different types of NPs. Protein loading of the heme-albumin loaded PDA NPs can be seen in the SEM images as the surface texture between unloaded and loaded PDA NPs has changed. DLS results are presented with a Gaussian distribution in FIG. 13. Zeta potential of both nanoparticle types was investigated and shown in Table 2.

TABLE 2
Zeta Potential of PDA NPs with and without incorporation
of the home-albumin protein complex.

| | Zeta Potential (mV) | Standard Deviation (mV) |
|---|---|---|
| Unloaded | −31.10 | ±5.44 |
| Loaded | −44.41 | ±3.42 |

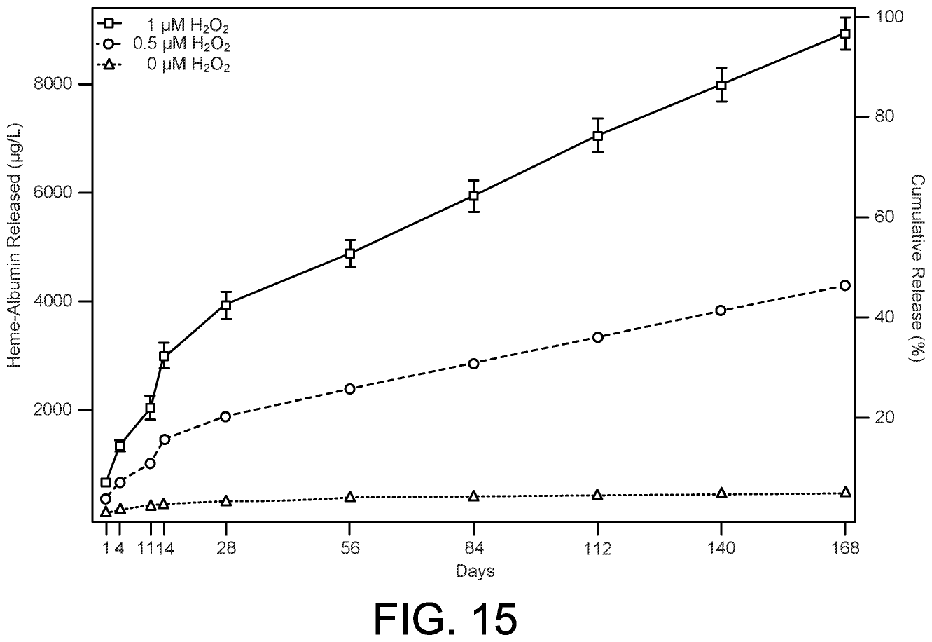

In Vitro Release of Heme-Albumin. In vitro extended release of heme-albumin from PDA NPs was investigated at varying concentrations of oxidizing agent using 0, 0.5, and 1 mM H₂O₂ as a model for the oxidative stress conditions present in AMD. At time points of 1, 4, 11, 14, 28, 56, 84, 112, 140, and 168 days, NPs were collected and total protein in the supernatant was measured by the BCA assay. The results are presented in FIG. 15. At all concentrations of H₂O₂, heme-albumin loaded PDA NPs provided consistent and sustained release of heme-albumin. More protein was released at higher oxidative stress levels, as expected, providing tunable therapeutic dosing for the changing oxidative stress conditions present in AMD. Cumulative release of the protein at 140 days at 0.5 and 1 mM H₂O₂ was 4301.6±54.8 μg/mL and 8942.4±295.6 μg/mL, respectively. Compared to published uses of PDA NPs, where it has been demonstrated that PDA NPs provided up to 6 months of extended delivery of model protein BSA, our study confirms the ability of the PDA NPs to provide sustained and ROS-responsive release of new protein therapeutics such as the heme-albumin complex.⁴²

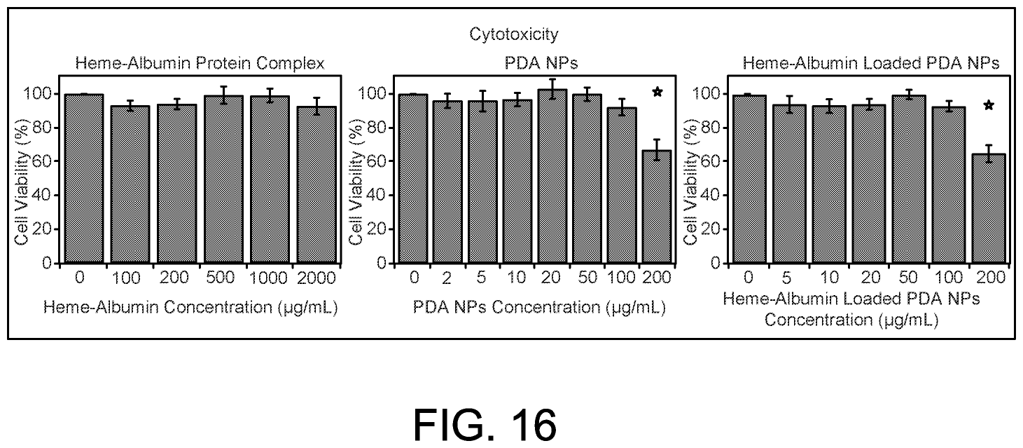

Cytotoxicity of Heme-Albumin, Heme-Albumin loaded PDA NPs, and Blank NPs. Biocompatibility is an important consideration for determining the therapeutic use of any developed drug or drug delivery system. The synthesized heme-albumin protein complex, and both unloaded and loaded PDA NPs, were investigated with retinal pigment epithelial cells for understanding the interactions with one of the most impacted cell types present in AMD. The toxicity of heme-albumin as a therapeutic has yet to be determined and was investigated at varying concentrations. Between 100-2000 μg/mL, heme-albumin showed no significant cytotoxicity to the dosed ARPE-19 cells after 24-hour incubation and MTS staining. The LC₅₀ of the therapeutic is >2000 μg/mL, showing potential for a wide range of available therapeutic doses. Further, cytotoxicity of blank and heme-albumin loaded PDA NPs were evaluated using the same methods. ARPE-19 cells were incubated with varying concentrations of either blank, non-protein loaded NPs, or heme-albumin loaded PDA NPs, ranging from 2-200 μg/mL, and stained with MTS dye 24 hours after initial dosing. As shown in FIG. 16, both loaded and unloaded PDA NPs were found to have minimal cytotoxicity (p<0.05) to the retinal cells at concentrations less than 200 μg/mL.

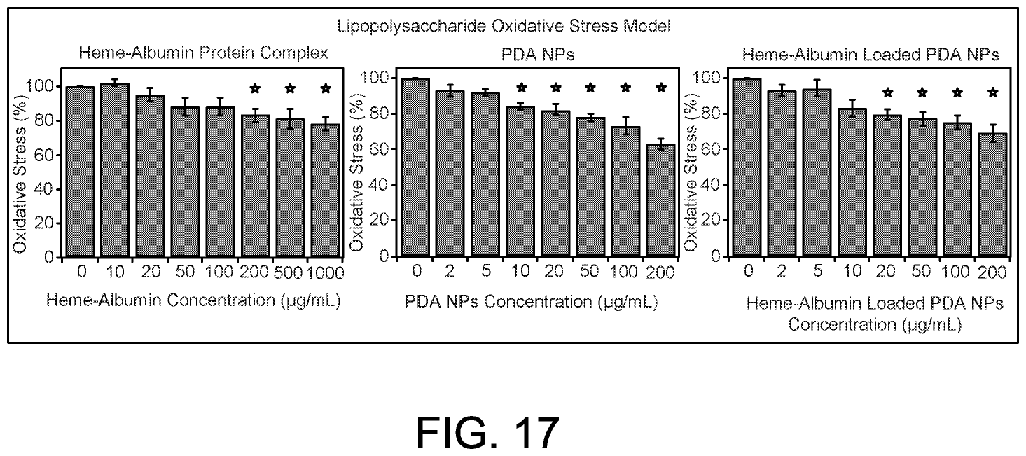

Oxidative Stress Reduction in Inflammatory Model. Chronic inflammation is a hallmark of dry AMD and plays a crucial role in disease progression to geographic atrophy and wet AMD. The constant oxidative injury that occurs with continued immune signalling leads to cellular fatigue and tissue damage within the retina and affected surrounding regions. The ability of heme-albumin to combat oxidative stress in an inflammatory model was investigated. A concentration of 10 μg/mL of LPS was applied to the ARPE-19 cells in conjunction with varying concentrations of the therapeutic groups. After 24 hrs, the cells were stained with 10 μM DCFDA stain for 45 minutes before fluorescence was measured at Ex/Em=485/535 nm. The results are shown in FIG. 17. Heme-albumin was able to provide significant reduction in oxidative stress, when compared to the control at all therapeutic concentrations above 200 μg/mL to a maximum reduction of 22±10% at 1000 μg/mL. Both nanoparticle types, unloaded PDA NPs and heme-albumin loaded PDA NPs were able to significantly combat oxidative stress in LPS inflammatory model.

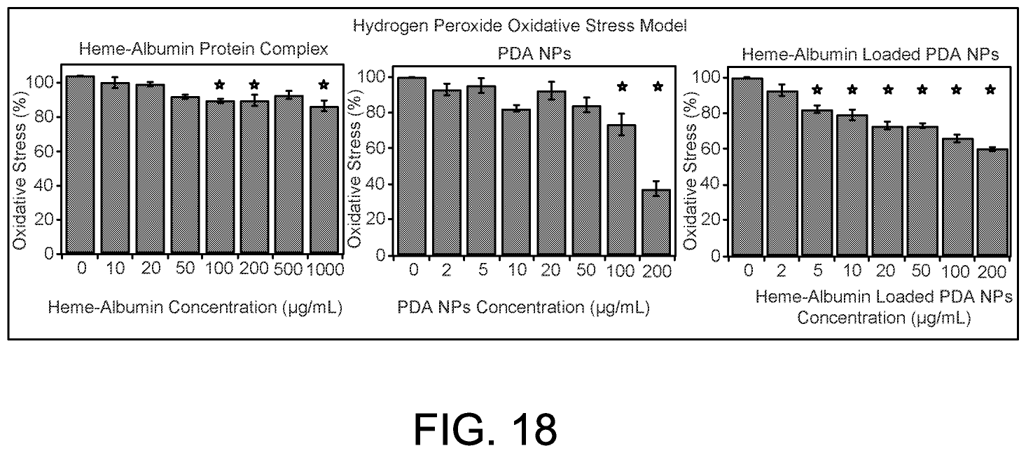

Oxidative Stress Reduction in H₂O₂ ROS Model. With aging and metabolic fatigue, the imbalance that occurs within the posterior segment of the eye results in accumulation of ROS, causing tissue damage and overexpression of inflammatory cytokines and growth factors. The ability of heme-albumin, unloaded PDA NPs, and heme-albumin loaded PDA NPs to reduce oxidative stress from ROS was therefore investigated. ARPE-19 cells were challenged with 100 μM H₂O₂ and simultaneously treated with varying concentrations of therapeutics. As shown in FIG. 18, heme-albumin, at a concentration of 1000 μg/mL, reduced oxidative stress by 17±3% (p=0.005) as compared to control which experienced no oxidative challenge or therapeutic dosing.

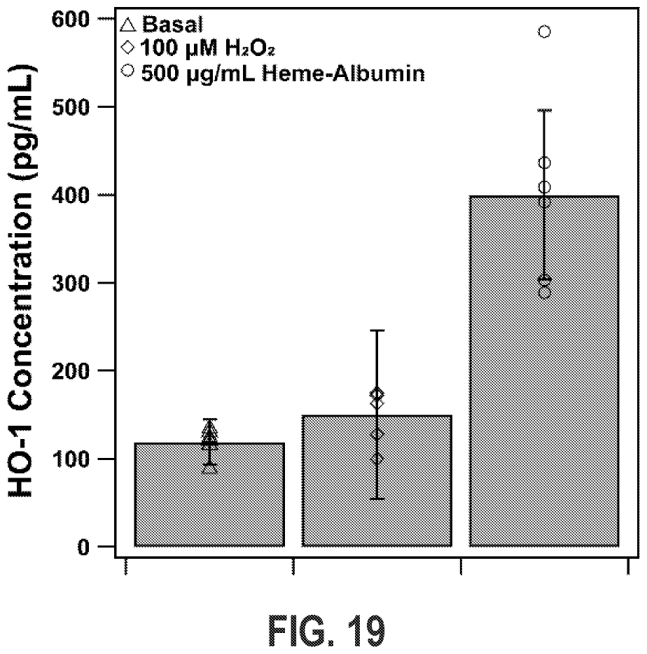

Heme-Oxygenase 1 Expression is Increased by Heme-Albumin. The proposed therapeutic activity of heme-albumin is provided by induction of the HO-1 enzyme that, through catabolismof heme, generates the anti-inflammatory and antioxidant components CO and biliverdin. Expression of HO-1 by exposure to 500 μg/mL heme-albumin was quantified by ELISA, at both baseline and stressed conditions, as depicted in FIG. 19. Compared to untreated control, there was a significant increase in the expression of HO-1 (p=0.002) in RPE cells from 118.9±14.9 pg/mL to 274.0±44.3 pg/mL at baseline conditions. Further expression of HO-1 was shown under stressed conditions where, compared to the oxidatively challenged control, HO-1 expression increased from 135.9±25.2 pg/mL to 381.1±96.33 pg/mL, validating the potential of the proposed treatment pathway for the therapeutic protein complex.

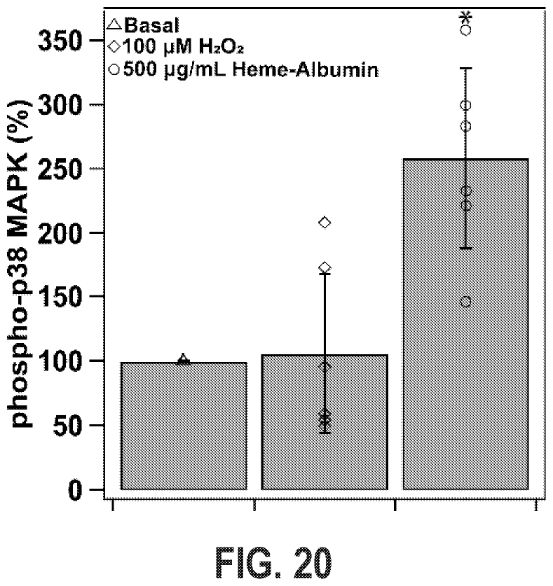

Heme-Albumin increases phospho-p38 MAPK Expression. Induction of HO-1 and production of CO by-product is theorized to provide anti-inflammatory relief through cytokine regulation through the p38 MAPK pathway. Heme-albumin's ability to increase expression of activated p38 MAPK was measured through ELISA and was shown to induce a statistically significant (p<0.05) in ARPE-19 (FIG. 20). Compared to basal condition control, heme-albumin caused a 200% increase in activated protein expression.

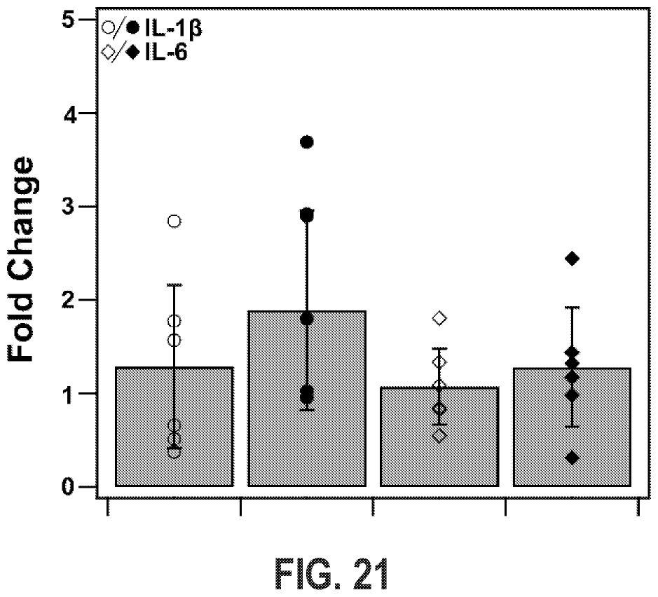

Heme-albumin does not induce pro-inflammatory cytokine response without activation of p38 MAPK. To determine heme-albumin's impact on inflammatory signaling in unstressed conditions, PCR analysis was conducted on pro-inflammatory cytokines, IL-1β and IL-6. Cytokines were measured and compared to untreated control. Results are shown in FIG. 21. Treatments of 500 μg/mL heme-albumin without oxidative challenge do not induce additional cytokine expression as compared to untreated control.

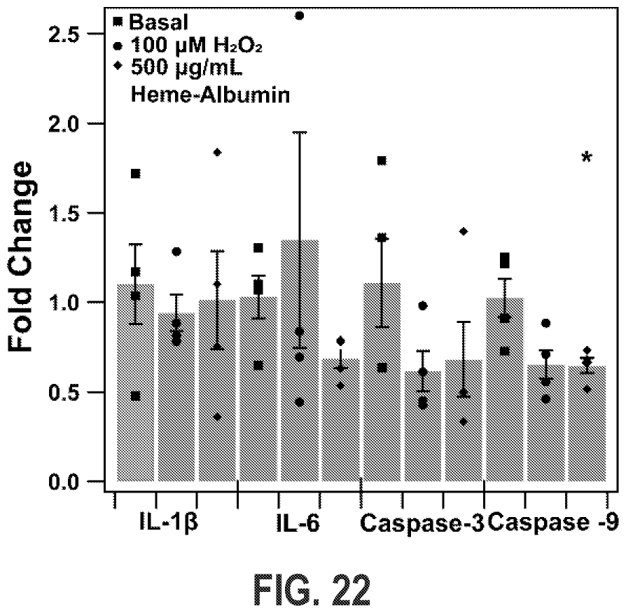

Treatment of heme-albumin in oxidative stress conditions reduces pro-apoptotic and IL-6 gene expression. The impact of heme-albumin on pro-inflammatory and pro-apoptotic gene expression in oxidative stress conditions was determined and results are presented in FIG. 22. In both interleukins, there was not a statistically significant expression of Il-1β or IL-6 nor was there a statistical difference in pro-apoptotic caspase-3 gene expression. Lastly, the pro-apoptotic gene caspase-9 did show a statistically significant difference (p=0.0468) compared to untreated control, showing that heme-albumin is able to reduce pro-apoptotic mRNA expression in oxidative stress conditions.

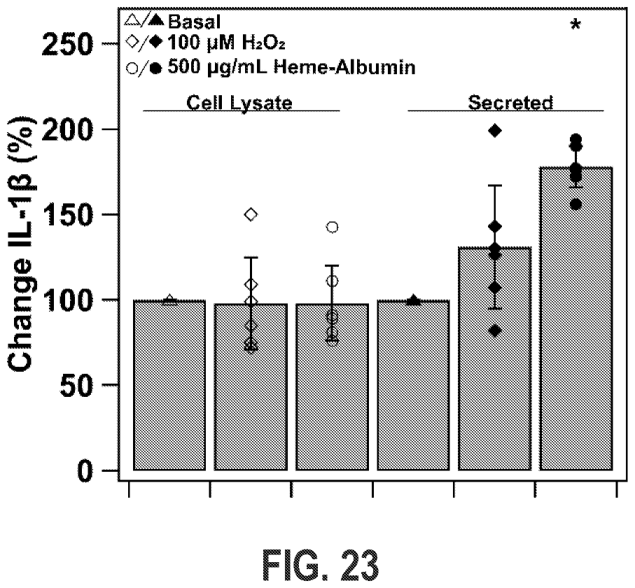

Heme-Albumin impacts IL-1β expression with oxidative challenge. To investigate heme-albumin's impact on inflammatory signalling, ELISA quantification of expressed protein was conducted on pro-inflammatory cytokine, IL-1β. Cytokine expression of basal, oxidatively stressed and co-treatment of oxidative stress and heme-albumin in both cell lysate and media was evaluated. Results are shown in FIG. 23 as percent expression compared to control. Compared to basal control in cell lysate, neither 100 μM H₂O₂ challenge or co-treatment with oxidative challenge or heme-albumin caused a statistically significant change to IL-1 β expression. Secretion of IL-1β did show a statistically significant difference (p<0.05) compared to untreated control and is theorized to be related to the increase in protein expression of p38 MAPK.^{44,45}

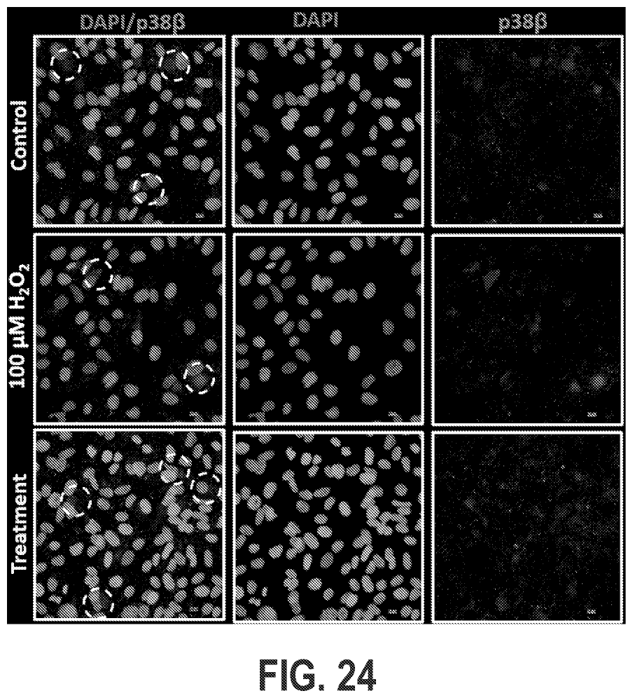

Heme-Albumin induces p38β MAPK isoform expression. Induction of HO-1 by heme-albumin for anti-inflammatory and antioxidant relief of oxidative stress activated further downstream expression of p38 MAPK Heme-albumin's ability to increase total p38 MAPK expression was demonstrated by immunofluorescence staining of ARPE-19 cells. This is shown in FIG. 24.

### Conclusion

The pathogenesis of AMD is complex and is exacerbated largely by the confounding oxidative stress of chronic inflammation and ROS affecting the central vision, causing tissue damage and inevitable blindness if left untreated. Treatment for dry AMD with antioxidant and glucocorticoid steroid therapies have shown potential benefits but long-term steroid use is often unfavorable due to their undesirable side effects, including cataract.⁴⁶ The current standard treatment for the ocular disorder involves up to monthly intravitreal injections that can result in poor patient compliance and treatment management, often leading to further vision loss. To improve treatment options, a novel heme-albumin protein complex delivered by sustained release from polydopamine nanoparticles was investigated to address the inflammatory aspect underlying both forms of AMD. As a free molecule, heme is a low molecular weight and hydrophobic molecule that readily entraps itself in lipophilic cell membranes and lipoproteins, but when bound to human serum albumin (HSA), an important serum protein capable of carrying a variety of types of cargo, heme's hydrophobicity and cytotoxicity are mitigated. The synthesis of both heme-albumin protein complex and loaded PDA nanoparticles was performed to address inflammation and ROS present in AMD. Morphology and zeta potential of the heme-albumin loaded PDA NPs and unloaded PDA NPs were characterized and the in vitro release of heme-albumin at varying concentrations of H₂O₂ from protein loaded PDA NPs were quantified over a 6-month period, demonstrating sustained release for several months. Higher concentrations of H₂O₂ induced release of heme-albumin, providing increased therapeutic potential against deleterious levels of ROS. Heme-albumin showed no significant cytotoxicity to ARPE-19 cells up to 2000 μg/mL whereas both unloaded and heme-albumin loaded PDA NPs showed minimal cytotoxicity up to concentrations of 200 μg/mL. Heme-albumin was able to (p=0.005) reduce oxidative stress in both inflammatory and ROS models with reductions of up to 22% and 17%, respectively. Additionally, the novel protein therapeutic caused a significant difference in the expression of HO-1, an essential enzyme in the immune stress response which is vital for therapeutic action. With the oxidative stress elevated by AMD development, HO-1 would be highly expressed to provide immune support. Delivery of heme-albumin loaded PDA NPs could therefore potentially provide sustained delivery of heme-albumin to further induce the anti-inflammatory and antioxidant benefits of HO-1 Incorporated into the delivery system, heme-albumin loaded PDA NPs showed a maximum reduction of oxidative stress of 25% and 34% (p<0.001) for inflammatory and ROS models, respectively. Compared to traditional, frequent intravitreal administration of anti-VEGF and oral administration of high dose antioxidants for treatment of wet and dry AMD, heme-albumin loaded PDA NPs have potential for reduced injection frequency and a dual method of combating the causes of oxidative assault for innovative treatment of AMD. Future studies will explore safety and efficacy of this therapeutic approach in in vivo models of AMD.

### REFERENCES

1. World Health Organisation, Global Data on Visual Impairment 2010, 2010, 17.
2 The Lancet Global Health: Vision loss could be treated in one billion people worldwide, unlocking human potential and accelerating global development | Michigan Medicine, https://www.uofmhealth.org/news/archive/202102/lancet-global-health-vision-loss-could-be-treated-one, (accessed 9 Nov. 2021).
3. Age-Related Macular Degeneration (AMD) Data and Statistics | National Eye Institute, https://www.nei.nih.gov/learn-about-eye-health/outreach-campaigns-and-resources/eye-health-data-and-statistics/age-related-macular-degeneration-amd-data-and-statistics, (accessed 31 Aug. 2021).
4. J. Ambati and B. J. Fowler, Neuron, 2012, 75, 26-39.
5. N. M. Schultz, S. Bhardwaj, C. Barclay, L. Gaspar and J. Schwartz, Clin Ther, 2021, 43, 1792-1818.
6. R. P. Danis, J. A. Lavine and A. Domalpally, Clin Ophthalmol, 2015, 9, 2159.
7. R. Mahmoudzadeh, J. W. Hinkle, J. Hsu and S. J. Garg, Curr Opin Ophthalmol, 2021, 32, 294-300.
8. A. Gheorghe, L. Mahdi and O. Musat, Rom J Ophthalmol, 2015, 59, 74.
9 H. E. Grossniklaus and W. R. Green, Am J Ophthalmol, 2004, 137, 496-503.
10. T. J. Heesterbeek, L. Lor Es-Motta, C. B. Hoyng, Y. T. E. Lechanteur and A. I. Den Hollander, Ophthalmic Physiol Opt, 2020, 40, 140-170.
11. L. F. Hernández-Zimbrón, R. Zamora-Alvarado, L. Ochoa-De La Paz, R. Velez-Montoya, E. Zenteno, R. Gulias-Cañizo, H. Quiroz-Mercado and R. Gonzalez-Salinas, Oxid Med Cell Longev, 2018, 8374647.
12. S. G. Jarrett and M. E. Boulton, Mol Aspects Med, 2012, 33, 399-417.
13. L. J. Rohowetz, J. G. Kraus and P. Koulen, Int J Mol Sci, DOI:10.3390/ijms19113362.
14. M. Nita and A. Grzybowski, Oxid Med Cell Longev,, DOI:10.1155/2016/3164734.
15. P. S. Mettu, A. R. Wielgus, S. S. Ong and S. W. Cousins, Mol Aspects Med, 2012, 33, 376-398.
16. D. V. Telegina, O. S. Kozhevnikova and N. G. Kolosova, Biochemistry (Moscow), 2018, 83, 1009-1017.
17. A. G. Smith and P. K. Kaiser, Expert Opin Emerg Drugs, 2014, 19, 157-164.
18. J. G. Garweg, J. J. Zirpel, C. Gerhardt and I. B. Pfister, Graefe's Archive for Clinical and Experimental Ophthalmology, 2018, 256, 823-831.
19. K. G. Falavarjani, Q. D. Nguyen, K. Ghasemi Falavarjani and Q. D. Nguyen, 2013,27, 787-794.
20. A. Sharma, N. Parachuri, N. Kumar, B. D. Kuppermann and F. Bandello, Eye 2021, 2021, 1-2.
21. M. Gemenetzi, A. J. Lotery and P. J. Patel, Eye 2017, 2016, 31, 1-9.
22. H. J. Cho, S. G. Yoo, H. S. Kim, J. H. Kim, C. G. Kim, T. G. Lee and J. W. Kim, Am J Ophthalmol, 2015, 159, 285-292.e1.
23. T. Antonio Cabral de Guimaraes, M. Daich Varela, M. Georgiou, M. Michaelides, D. M. Varela and al J. Br Ophthalmol, Br J Ophthalmol, 2022, 106, 297-304.
24. A. Sadaka and G. P. Giuliari, Clinical Ophthalmology, 2012, 6, 1325-1333.
25. A. A. Waza, Z. Hamid, S. Ali, S. A. Bhat and M. A. Bhat, Inflammation Research, 2018, 67, 579-588.
26. R. Gozzelino, V. Jeney and M. P. Soares, The Annual Review of Pharmacology and Toxicology is Annu. Rev. Pharmacol. Toxicol, 2010, 50, 323-354.
27. G. Silva, A. Cunha, I. P. Grégoire, M. P. Seldon and M. P. Soares, The Journal of Immunology, 2006, 177, 1894-1903.
28. A. D. Bachstetter and L. J. Van Eldik, Aging Dis, 2010, 1, 199.
29 C. Mancuso, Free Radic Biol Med, 2021, 172, 521-529.
30. W. Chen, G. J. Maghzal, A. Ayer, C. Suarna, L. L. Dunn and R. Stocker, Free Radic Biol Med, 2018, 115, 156-165.
31. B. Wegiel and L. E. Otterbein, Front Pharmacol, 2012, 3 Mar., 47.
32. R. Krishnan Kutty, C. N. Nagineni, G. Kutty, J. J. Hooks, G. J. Chader and B. Wiggert, J Cell Physiol, 1994, 159, 371-378.
33. J. Kuesap, B. Li, S. Satarug, K. Takeda, I. Numata, K. Na-Bangchang and S. Shibahara, Biochem Biophys Res Commun, 2008, 367, 413-419.
34. M. Taverna, A. L. Marie, J. P. Mira and B. Guidet, Ann Intensive Care, 2013, 3, 1-7.
35. M. M. Allyn, R. H. Luo, E. B. Hellwarth and K. E. Swindle-Reilly, Front Med (Lausanne), 2021, 8, 787644.
36. H. Zhao, Z. Zeng, L. Liu, J. Chen, H. Zhou, L. Huang, J. Huang, H. Xu, Y. Xu, Z. Chen, Y. Wu, W. Guo, J. H. Wang, J. Wang and Z. Liu, Nanoscale, 2018, 10, 6981-6991.
37. X. Bao, J. Zhao, J. Sun, M. Hu and X. Yang,, DOI:10.1021/acsnano.8b04022.
38. S. Jia, S. Dong, H. Liu, H. Yu, Z. Chen, S. Wang, W. Li, R. Peng, F. Li, Q. Jiang and J. Liu, Biomater Sci, DOI:10.1039/D1BM02026A.
39. T. Kostka, J. Fohrer, C. Guigas, Karlis Briviba, N. Seiwert, J. Fahrer, P. Steinberg and M. T. Empl, 2020, 94, 3911-3927.
40. R. J. Wong, H. J. Vreman, S. Schulz, F. S. Kalish, N. W. Pierce and D. K. Stevenson, Journal of Perinatology 2011 31:1, 2011, 31, S35-S41.
41. F. Vallelian, C. A. Schaer, J. W. Deuel, G. Ingoglia, R. Humar, P. W. Buehler and D. J. Schaer, Pharmacol Res Perspect, DOI:10.1002/PRP2.392.
42. P. Jiang. A. Choi and K. E. Swindle-Reilly, Nanoscale, 2020, 12, 1-33.
43. M. Anraku, R. Shintomo, K. Taguchi, U. Kragh-Hansen, T. Kai, T. Maruyama and M. Otagiri, Life Sci, 2015, 134, 36-41.
44. J. J. Baldassare, Y. Bi and C. J. Bellone, J Immunol, 1999, 162, 5367-5373.
45. J Taylor, D. L. Dewitt, J. Saklatvala, S. H. Ridley, S. J. Sarsfield, J. C. Lee, H. F. Bigg and T. E. Cawston, J Immunol., 1997, 158, 3165-3173.
46. R. S. Sulaiman, M. Kadmiel and J. A. Cidlowski, Steroids, 2018, 133, 60-66.

The compositions and methods of the appended claims are not limited in scope by the specific compositions and methods described herein, which are intended as illustrations of a few aspects of the claims. Any compositions and methods that are functionally equivalent are intended to fall within the scope of the claims. Various modifications of the compositions and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A method of treating an ophthalmological disorder in a subject in need thereof comprising contacting the eye of the subject a therapeutically effective amount of an ocular therapeutic composition comprising heme.

2. The method of claim 1, wherein the heme comprises a heme conjugate.

3. The method of claim 2, wherein the heme conjugate comprises heme-human serum albumin (heme-HSA).

4. The method of claim 2, wherein the heme conjugate comprises methemoglobin.

5. The method of claim 2 or 4, wherein the heme conjugate comprises a methemoglobin-baptoglobin conjugate.

6. The method of claim 4, wherein the methemoglobin is conjugated to human serum albumin (HSA).

7. The method of claim 4, wherein the heme conjugate comprises polymerized methemoglobin.

8. The method of any of claims 1-7, wherein the ocular therapeutic composition further comprises a delivery vehicle for delivery of the heme to the eye.

9. The method of claim 8, wherein the delivery vehicle comprises a population of particles formed from a biocompatible polymer, and wherein the heme is associated with the particles.

10. The method of claim 9, wherein the heme is encapsulated within the particles.

11. The method of any of claims 9-10, wherein the heme is non-covalently associated with the particles, such as adsorbed to the surface of the particles.

12. The method of any of claims 9-11, wherein the biocompatible polymer comprises a biodegradable polymer.

13. The method of any of claims 9-12, wherein the biocompatible polymer comprises polydopamine.

14. The method of any of claims 9-13, wherein the population of particles have an average particle size of from about 10 nm to about 1000 nm, such as from about 100 nm to about 400 nm, from about 100 nm to about 200 nm, from about 120 nm to about 270 nm, or from about 120 nm to about 170 nm.

15. The method of any of claims 9-14, wherein the population of particles has an average particle size of about 150 nm, about 175 nm, or about 200 nm.

16. The method of any of claims 9-15, wherein the beme is released upon exposure of the particles to reactive oxygen species.

17. The method of any of claims 9-16, wherein each of the particles are further coated with a coating polymer.

18. The method of any of claims 1-17, wherein the ocular therapeutic composition further comprises an additional active agent.

19. The method of claim 18, wherein the additional active agent comprises an ophthalmic drug, such as an anti-glaucoma agent, an anti-angiogenesis agent, an anti-vascular endothelial growth factor (VEGF) agent, an anti-infective agent, an anti-inflammatory agent, a growth factor, an immunosuppressant agent, an anti-allergic agent, a complement inhibitor, or any combinations thereof.

20. The method of any of claims 18-19, wherein the ocular therapeutic composition further comprises a delivery vehicle for delivery of the heme to the eye, and wherein the delivery vehicle further provides for delivery of the additional active agent to the eye.

21. The method of claim 20, wherein the delivery vehicle comprises a population of particles formed from a biocompatible polymer, and wherein the additional active agent is associated with the particles, such as encapsulated within the particles and/or non-covalently associated with the particles, such as adsorbed to the surface of the particles.

22. The method of any of claims 1-21, wherein the ophthalmological disorder is acute macular neuroretinopathy; Behcet's disease; neovascularization, including choroidal neovascularization; diabetic uveitis; histoplasmosis; infections, such as fungal or viral-caused infections; macular degeneration, such as acute macular degeneration (AMD), including wet AMD, non-exudative AMD and exudative AMD; retinal degenerative diseases such as geographic atrophy, edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic ophthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, nonretinopathy diabetic retinal dysfunction, retinitis pigmentosa, a cancer, and glaucoma.

23. The method of claim 22, wherein the ophthalmological disorder is AMD, such as dry AMD.

24. The method of any one of claims 1-23, wherein contacting the eye of the subject comprises topically applying the ocular therapeutic composition to the eye of the subject.

25. The method of any one of claims 1-23, wherein contacting the eye of the subject comprises injecting the ocular therapeutic composition into the eye of the subject.

26. The method of claim 25, wherein injecting into the eye of the subject comprises injecting the ocular therapeutic composition into the vitreous chamber of the eye.

27. The method of any one of claims 25-26, wherein injecting into the eye of the subject comprises an intravitreal injection, a subconjunctival injection, a subtenon injection, a retrobulbar injection, or a suprachoroidal injection.

28. An ocular therapeutic composition comprising a therapeutically effective amount of heme to treat or prevent an ophthalmological disorder in a subject in need thereof.

29. The composition of claim 28, wherein the heme comprises a heme conjugate.

30. The composition of claim 29, wherein the heme conjugate comprises heme-human serum albumin (heme-HSA).

31. The composition of claim 29, wherein the heme conjugate comprises methemoglobin.

32. The composition of claim 29 or 31, wherein the heme conjugate comprises a methemoglobin-haptoglobin conjugate.

33. The composition of claim 31, wherein the methemoglobin is conjugated to human serum albumin (HSA).

34. The composition of claim 31, wherein the heme conjugate comprises polymerized methemoglobin.

35. The composition of any of claims 28-34, wherein the ocular therapeutic composition further comprises a delivery vehicle for delivery of the heme to the eye.

36. The composition of claim 35, wherein the delivery vehicle comprises a population of particles formed from a biocompatible polymer, and wherein the heme is associated with the particles.

37. The composition of claim 36, wherein the heme is encapsulated within the particles.

38. The composition of any of claims 36-37, wherein the heme is non-covalently associated with the particles, such as adsorbed to the surface of the particles.

39. The composition of any of claims 36-38, wherein the biocompatible polymer comprises a biodegradable polymer.

40. The composition of any of claims 36-39, wherein the biocompatible polymer comprises polydopamine.

41. The composition of any of claims 36-40, wherein the population of particles have an average particle size of from about 10 nm to about 1000 nm, such as from about 100 nm to about 400 nm, from about 100 nm to about 200 nm, from about 120 nm to about 270 nm, or from about 120 nm to about 170 nm.

42. The composition of any of claims 36-41, wherein the population of particles has an average particle size of about 150 nm, about 175 nm, or about 200 nm.

43. The composition of any of claims 36-42, wherein the heme is released upon exposure of the particles to reactive oxygen species.

44. The composition of any of claims 36-43, wherein each of the particles are further coated with a coating polymer.

45. The composition of any of claims 28-44, wherein the ocular therapeutic composition further comprises an additional active agent.

46. The composition of claim 45, wherein the additional active agent comprises an ophthalmic drug, such as an anti-glaucoma agent, an anti-angiogenesis agent, an anti-vascular endothelial growth factor (VEGF) agent, an anti-infective agent, an anti-inflammatory agent, a growth factor, an immunosuppressant agent, an anti-allergic agent, a complement inhibitor, or any combinations thereof.

47. The composition of any of claims 45-46, wherein the ocular therapeutic composition further comprises a delivery vehicle for delivery of the heme to the eye, and wherein the delivery vehicle further provides for delivery of the additional active agent to the eye.

48. The composition of claim 47, wherein the delivery vehicle comprises a population of particles formed from a biocompatible polymer, and wherein the additional active agent is associated with the particles, such as encapsulated within the particles and/or non-covalently associated with the particles, such as adsorbed to the surface of the particles.


SEQUENCE LISTING (8 sequences)

SEQ ID NO: 1 - DNA, 19 bp, synthetic construct
  source 1..19: mol_type = other DNA
tcggtccagttgccttctc

SEQ ID NO: 2 - DNA, 19 bp, synthetic construct
  source 1..19: mol_type = other DNA
gaggtgagtggctgtctgt

SEQ ID NO: 3 - DNA, 21 bp, synthetic construct
  source 1..21: mol_type = other DNA
ctcgccagtgaaatgatggct

SEQ ID NO: 4 - DNA, 21 bp, synthetic construct
  source 1..21: mol_type = other DNA
gtcggagattcgtagctggat

SEQ ID NO: 5 - DNA, 22 bp, synthetic construct
  source 1..22: mol_type = other DNA
gtttgaggaccttcgaccagct

SEQ ID NO: 6 - DNA, 22 bp, synthetic construct
  source 1..22: mol_type = other DNA
caacgtaccaggagccactctt

SEQ ID NO: 7 - DNA, 23 bp, synthetic construct
  source 1..23: mol_type = other DNA
ggaagcgaatcaatggactctgg

SEQ ID NO: 8 - DNA, 22 bp, synthetic construct
  source 1..22: mol_type = other DNA
gcatcgacatctgtaccagacc